(19) United States
(12) Patent Application Publication
(10) Pub. No.: US 2003/0084468 A1
(43) Pub. Date: May 1, 2003

(54) Methods of expressing transgenes

(76) Inventors: Aris N. Economides, Tarrytown, NY (US); Thomas M. DeChiara, Paramus, NJ (US); Nicholas W. Gale, Tarrytown, NY (US); George D. Yancopoulos, Yorktown Heights, NY (US)

Correspondence Address: Linda O. Palladino, Regeneron Pharmaceuticals, Inc., 777 Old Saw Mill River Road, Tarrytown, NY, 10591, US

(21) Appl. No.: 10/235,400

(22) Filed: Sep. 5, 2002

Related U.S. Application Data
(60) Provisional application No. 60/317,412, filed on Sep. 5, 2001.

(51) Int. Cl. A01K 67/027; C12Q 1/68; C12N 5/06; C12N 15/85

(52) U.S. Cl. 800/18; 800/21; 435/6; 435/455; 435/354; 435/320.1

(57) ABSTRACT

The present invention is a novel method of expressing transgenes in vivo by targeting protected transgene cassettes into predetermined loci, including ubiquitously expressed chromosomal loci, such that the activity of an exogenous promoter is maintained. The advantages of this method are that the expression pattern is determined primarily by the nature of the exogenous promoter and, therefore, is not subject to positional effects. The invention also encompasses the DNA targeting vectors, the targeted cells, as well as non-human organisms, especially mice, created from the targeted cells.

This application claims priority of U.S. Provisional Application No. 60/317,412, filed Sep. 5, 2001. Throughout this application various publications are referenced. The disclosures of these publications in their entireties are hereby incorporated by reference into this application.

### FIELD OF THE INVENTION

The field of this invention is a method of expressing transgenes and evaluating their activity by targeting DNA vectors into predetermined loci, including ubiquitously expressed loci, such as the ROSA 26 locus. The field of the invention also encompasses DNA targeting vectors, targeted cells, as well as non-human organisms, such as mice, created from the targeted cells.

### BACKGROUND OF THE INVENTION

Transgenic and knockout (KO) animals are used extensively to gain insight into gene function and to evaluate drug-target candidate genes and novel protein-based therapeutics in whole organisms. In the case of KO animals, the gene of interest is usually replaced by a marker gene to create a heterozygous null allele that can then be bred to homozygosity. A homozygous null allele may lead to a phenotype that can be used to understand the function of the gene of interest in vivo. However, about 60% of homozygous null allele mutant animals do not exhibit a phenotype and, if they do exhibit a phenotype, the phenotype only supplies information as to what happens when the gene of interest is absent. Therefore in order to gain a more complete understanding of the functions of a gene and in order to evaluate its potential as a drug-target candidate gene, a complimentary approach is often utilized in which a gene of interest is over-expressed and/or miss-expressed by engineering transgenic animals. In transgenic animals, depending on how the DNA vector or vector carrying the transgene is designed, the gene of interest can be over-expressed (i.e. expressed at levels higher that those normally produced by the wild type gene), miss-expressed (i.e. expressed in a tissue different from the tissue or tissues in which it is normally expressed and/or at a time that is not normally expressed), or both. Importantly, it should be noted that the expression levels and expression profiles depend, to a large extent, on the choice of promoter driving the transgene. Furthermore, transgenic animal technology can be used to express any conceivable version of the gene of interest, including but not limited to mutant and tagged forms, without affecting the activity of the normal endogenous copies of the gene of interest. Combined with the ability to turn expression of the transgene either on or off at specific points in time or under certain sets of conditions (for example, by using regulated Cre or related technologies (Kellendonk et al., 1996, Nucleic Acids Res, 24, 1404-11; Nagy and March, 2001, Methods Mol Biol, 158, 95-106; Nichols et al., 1997, Mol Endocrinol, 11, 950-61; Rossant and McMahon, 1999, Genes Dev, 13, 142-5.; Schwenk et al., 1998, Nucleic Acids Res, 26, 1427-32; Vooijs et al., 2001, EMBO Rep, 2, 292-297), Tet-regulated systems (Baron and Bujard, 2000, Methods Enzymol, 327, 401-21; Blau and Rossi, 1999, Proc Natl Acad Sci USA, 96, 797-9.; Gossen and Bujard, 1992, Proc Natl Acad Sci USA, 89, 5547-51.; Gossen et al., 1995, Science, 268, 1766-9.; Shockett and Schatz, 1996, Proc Natl Acad Sci USA, 93, 5173-6), or other suitable technology familiar in the art), it is possible to carefully dissect the in vivo functions of a gene of interest and to evaluate drug-target candidate genes and novel protein-based therapeutics.

In spite of the advantages and utility of transgenic animal technology, currently available methods for creating transgenic animals suffer from several significant technical problems. The most frequently utilized method for creating a transgenic mouse is pronuclear injection (Jackson and Abbot, 2000, The Practical Approach Series, 299). In this method, a DNA vector carrying the gene of interest is inserted downstream of a promoter and is followed by a polyadenylation signal sequence (FIG. 1). The promoter is generally chosen on the basis of its tissue specificity. In some instances, it is desirable to use an ubiquitous promoter (i.e. one that drives expression in many, if not all, the different tissues and cell types in the body), whereas in other instances it is desirable to use a tissue-specific promoter (i.e. one that drives expression in only one or a few tissues, the extreme example being a promoter that drives expression only in a single cell type, such as the insulin promoter which drives expression in the β-cells of pancreatic islets). The DNA vector is injected into oocytes that are subsequently implanted into foster or surrogate mothers. Once founder pups are born they are screened for expression of the transgene. Some of the more serious problems associated with this method arise from the fact that the introduced DNA vector integrates randomly and frequently in multiple copies into the genome. In turn, this random integration can often lead to several subsequent problems that become apparent upon examination of the founders such as:

Positional effects: Aberrant expression of the transgene (i.e. expression of the transgene that does not reflect the activity of the promoter chosen) is frequently observed (Bronson et al., 1996, Proc Natl Acad Sci USA, 93, 9067-72.; Freundlieb et al., 1999, J Gene Med, 1, 4-12.; Hatada et al., 1999, J Biol Chem, 274, 948-55.; Jackson and Abbot, 2000, The Practical Approach Series, 299). This can result from integration within or near a locus that contains regulatory elements that act on the promoter of the transgene cassette and modify the expression of the transgene so that its expression pattern no longer accurately reflects the expression pattern expected for that promoter. Positional effects are a problem for ubiquitous and tissue-specific promoters. To create transgenic animals wherein ubiquitous expression of the gene of interest is desired a ubiquitous promoter is used to drive expression of the transgene. However, it is often found that integration of the DNA vector within or near a locus that contains regulatory elements restricts the expression of the gene of interest to only a subset of tissues. Similar problems are frequently encountered when using a tissue-specific promoter to drive expression of a transgene. Often, the tissue-specific promoter is affected by regulatory elements that act on the site of integration, resulting in an expression pattern or profile that is different from that expected for the tissue-specific promoter. Although positional effects can be minimized by using BAC-based transgenic animal technologies (Jackson and Abbot, 2000, The Practical Approach Series, 299; Yang et al., 1997, Nat Biotechnol, 15, 859-65; Yang et al., 1999, Nat Genet, 22, 327-35), this method still has the problems described below and, in addition, because a single BAC may contain multiple genes, making a BAC-based transgenic animal can result in generating transgenic animals that express not only the gene of interest, but also any neighboring gene that might reside on the BAC.

Silencing of the transgene: It has been reported in the literature that multiple integrations of the transgene can lead to silencing (Garrick et al., 1998, Nat Genet, 18, 56-9.; Henikoff, 1998, Bioessays, 20, 532-5.; Lau et al., 1999, Dev Dyn, 215, 126-38) and instability of the transgene (Schmidt-Kastner et al., 1996, Somat Cell Mol Genet, 22, 383-92). This effect can confound screening of founders (see below). Multiple integrations also result in uncertainty as to which of the inserted copies is expressed. If some of the inserted copies lie on different chromosomes they can desegregate upon breeding. This can result in some of the offspring expressing the transgene and some not expressing it, therefore necessitating screening of the offspring until a stable line with the desired phenotype is identified.

Insertional inactivation of an endogenous allele: It has also been reported in the literature that insertion of a DNA vector can unintentionally inactivate or alter the expression pattern of an endogenous gene (Merlino et al., 1991, Genes Dev, 5, 1395-406). Although this may not be a problem if the transgenic animals are maintained as heterozygotes, it confounds breeding steps. Furthermore, if the insertional inactivation is not detected it can confuse interpretation of a phenotype by attributing the phenotype to expression of the transgene when in fact it is due to the generation of a null for the gene where the DNA vector has inserted itself. It has been estimated that as many as 10% of random integrations result in insertional inactivation of genes located at the site of integration (Jackson and Abbot, 2000, The Practical Approach Series, 299). Such events are hard to discover prior to extensive phenotypic analysis, genotyping, and mapping and cloning of the affected locus (for example see (Dong et al., 2002, Genomics, 79, 777-84). Although one may characterize the site of the insertion by cloning sequences upstream and downstream of the transgene, it may be difficult to determine exactly where the transgene has integrated because the mouse genome has yet to be sequenced to completion. In addition, the integration event may disrupt a regulatory element and identification of exactly which genes this disruption affects is even more difficult.

Lethality of transgene: If the expression of the transgene is deleterious to embryonic development, the desired outcome of pronuclear injection is never obtained due to selection pressures against the transgenic embryos during development. While this usually does notpose a problem with marker gene transgenics it is a very frequent problem when attempting to express signaling molecules. Therefore, a very significant problem with traditional transgenic technology is the inability to derive some lines and to be able to study even developmental effects, because the line cannot be propagated.

Taken together, these problems result in an overall uncertainty in conclusively attributing the phenotype of transgenic animals derived by this method to the transgene's expression. Because of the above-described problems, for each gene of interest, at least several transgenic founder lines must be screened for the expression profile of the transgene. Frequently, screening many founders is required in order to identify a reasonable number that display the desired expression profile. Once the founders have been identified, they have to be expanded by breeding and then again multiple lines (i.e. lines arising from different founders) need to be analyzed phenotypically in order to ensure that the observed phenotype is not due to a positional effect or inactivation of an endogenous allele. Finally, there always remains the uncertainty that insertional inactivation of an endogenous locus may still have occurred, and this possibility may confound breeding to homozygosity and maintenance of transgenic lines as homozygotes.

Another method for creating transgenic animals utilizes embryonic stem (ES) cells (Pirity et al., 1998, Methods Cell Biol, 57, 279-93; Rossant et al., 1993, Philos Trans R Soc Lond B Biol Sci, 339, 207-15). Although it does not rely on pronuclear injection, it does rely on random integration of the DNA vector containing the gene of interest and thus it also is susceptible to some of the same problems described above (i.e. positional effects and insertional inactivation of the endogenous allele). More recently, the idea of creating a transgenic animal by introducing DNA vectors containing the gene of interest into a specific chromosomal locus has been explored. Two different types of insertions have been made. One type is introducing a ‘promoter-gene of interest-polyadenylation site cassette ’ into a specific chromosomal locus, such as the hprt locus (Evans et al., 2000, Physiol Genomics, 2, 67-75.; Hatada et al., 1999, J Biol Chem, 274, 948-55.; Wallace et al., 2000, Nucleic Acids Res, 28, 1455-64). There are several disadvantages to this specific approach. One disadvantage lies in the choice of the hprt locus for targeting because it is subject to X-linked inactivation. This complicates breeding steps, as female mice have to be bred to homozygosity for reliable transmission of a transcriptionally active transgene to their progeny. In addition, although the hprt locus has been used to target several different transgenic DNA vectors, not all of these vectors have shown the pattern of expression expected by the choice of promoter used to drive the transgene in these vectors (Hatada et al., 1999, J Biol Chem, 274,948-55). For example, the human haptoglobin gene does not retain fidelity of expression when introduced into the hprt locus and thus its expression pattern appears to be subject to modification in that locus. A similar disparity between the expected and observed expression pattern has been observed in experiments in which the tie-2 promoter was knocked into the hprt locus (Evans et al., 2000, Physiol Genomics, 2, 67-75). In contrast to the authors' interpretation that their knock-in faithfully reproduced the expression pattern of the tie-2 gene, a careful examination of the pattern of expression of the tie-2 gene using conventional methods reveals that the expression pattern observed by Evans, et al. is only in a subset of the cell types in which tie-2 is normally expressed (Maisonpierre et al., 1993, Oncogene, 8, 1631-7.; Motoike et al., 2000, Genesis, 28, 75-81.; Sato et al., 1993, Proc Natl Acad Sci USA, 90, 9355-8.; Schlaeger et al., 1997, Proc Natl Acad Sci USA, 94, 3058-63). Therefore, it appears that in at least some cases, insertion of a transgene cassette into the hprt locus does not guarantee the lack of positional effects. The possibility of positional effects on the transgene and the X-linked inactivation of the hprt locus confound the general applicability of introducing transgene cassettes into the hprt locus.

The other type of insertion involves introducing the gene of interest into a specific chromosomal locus, thus utilizing the regulatory elements of that locus to control gene expression. In this situation, the resulting locus is usually referred to as ‘knock-in’, and expression of the gene of interest should be most similar to that of the gene(s) expressed by the targeted locus. This method can be useful for making transgenics when it is desirable to have the expression of the transgene reflect the sites of expression of the targeted locus. Though in some situations this is indeed desirable, it is still limiting and also presents some problems. For example, knocking a transgene into an endogenous locus may lead to a heterozygous null for the gene(s) residing within that locus if the inserted transgene disrupts the expression of the gene at the locus. Therefore, the targeted locus must be carefully selected for lack of a hemizygous null phenotype (Lindsay et al., 2001, Nature, 410, 97-101.; Nutt and Busslinger, 1999, Biol Chem, 380, 601-11.; Nutt et al., 1999, Nat Genet, 21, 390-5.; Wilkie, 1994, J Med Genet, 31, 89-98). Moreover, special care should be taken in maintaining such transgenic lines as heterozygous carriers since breeding to homozygosity would lead to generation of a homozygous nulls at the locus where the transgene has been introduced, and thus may exhibit a phenotype unrelated to the presence of the transgene.

Although these two methods can usually provide a solution to the positional effects and random insertional inactivation of uncharacterized endogenous loci, alleviating the need to screen for founders, they retain other problems such as complicated breeding steps or insertional inactivation of endogenous chromosomal loci that exhibit phenotypes when bred to homozygosity. Therefore, a need still remains for methods of expressing transgenes in vivo that allows for the rapid, reproducible, efficient, and simple generation of transgenic animals, and that is devoid of the confounding issues that exist in currently available methods.

Wallace, et al. (Wallace et al., 2000, Nucleic Acids Res, 28, 1455-64), describe a method of identifying chromosomal sites permissive for transgene expression that involves random integration of an hprt mini-gene and lacZ-containing vector into hprt-deficient ES cells, and subsequently screening for clones exhibiting a desired phenotype (in this instance, appropriate regulation of lacZ expression in the ES cells). Although the method appears to be useful, it has several significant disadvantages compared to the method of Applicants' invention, notably the fact that the site in which random integration has occurred is unknown and completely uncharacterized, both in terms of location and biological function. Thus, the long-term effects of knocking genes into that site are unclear with respect to both the biological effects on the transgenic animals (phenotype) and to the pattern and levels of expression of the transgene.

Additionally, the authors report that their method is suitable for use with transgenes possessing relatively simple controlling elements, whereas the method of Applicants' invention is not limited in this respect. Finally, Applicants' method utilizes predetermined, well-characterized loci, including transcriptionally active and ubiquitously expressed loci, such as the ROSA26 and BT-5 loci, as opposed to the uncharacterized “permissive” or “neutral” site describe by Wallace, et al. that appears to be capable of allowing transcription, but which lacks the desired characteristic of being predetermined, well-characterized and predictably transcriptionally active and, if desired, ubiquitously expressed in virtually all cells.

### SUMMARY OF THE INVENTION

In accordance with the present invention, Applicants provide a novel method of expressing transgenes in vivo in which the expression of the transgene is primarily determined by the exogenous promoter and any other optional regulatory and/or accessory elements included in the transgene cassette, referred to herein as a “protected transgene cassette”. When the protected transgene cassette is targeted into predetermined loci, including ubiquitously expressed loci, it functions as an autonomous unit, meaning that it directs expression of the transgene(s) and any regulatory and/or accessory genes present in the protected transgene cassette without being influenced by the endogenous promoter in the targeted chromosomal locus, although the expression of the transgene may be affected by other elements present in the targeted locus such as enhancers and locus control regions. The advantages of this method are that a) the expression pattern, i.e. the types of cells or tissues in which the transgene is expressed, is largely determined by the nature of the exogenous promoter and, therefore, is not subject to positional effects arising from random integration into uncharacterized or undetermined loci and b) the same endogenous locus can be targeted using different “protected transgene cassettes” to generate multiple transgenic lines that are directly comparable (the only variant being the particular transgene, and not the targeted locus), thus allowing for easier phenotypic comparisons. Comparisons can be made either by varying the exogenous promoter and keeping the transgene the same or by varying the transgene and keeping the promoter the same. Thus, one can study the effect of expressing the same transgene in different tissues, or variants of that transgene in the same tissue (for example, an activated versus a dominant versus a wild type form of a kinase), or different transgenes in the same tissue(s), without having to worry that the observed phenotypic differences are due to positional effects. This alleviates the need to study multiple transgenic animal lines derived from independent founders for each protected transgene cassette. In addition, the technology can be easily adapted for inducible expression of the transgene by incorporating suitable methodology, such as Cre/loxP or tet-inducible systems. Finally, depending on the choice of the endogenous locus used for targeting, mice can be bred to homozygosity without the confounding issues present when the transgene cassette has integrated randomly into the genome.

In accordance with the present invention, Applicants have combined for the first time protected transgene cassettes, in which the exogenous promoter driving expression of the transgene is preceded by a transcription termination signal, with targeting into a predetermined locus, including an ubiquitously expressed locus that is transcriptionally active in nearly all cell types, to achieve several important advances in the field of generating transgenic animals, including but not limited to:

(a) Autonomous function of the protected transgene cassette (i.e. (i) control of transgene expression by the exogenous promoter and any other regulatory or accessory elements present within the protected transgene cassette and, therefore, transgene(s) expression patterns that reflect the activity of the exogenous promoter or, in cases where other regulatory and accessory elements have been included, transgene(s) expression patterns that reflect the activity of the exogenous promoter as modified by the aforementioned elements; and (ii) no influence of the targeted endogenous locus promoter on the expression pattern of the transgene;

(b) The capability to target the same endogenous locus with protected transgene cassettes to yield transgenic lines where the only variable is the protected transgene cassette of choice, and therefore empowering comparison of phenotypes resulting from each cassette, and where the possibility of contribution to the phenotype by insertional inactivation or positional effects has been eliminated;

(c) The capability to combine this method with previously described technology thus achieving nearly 100% targeting frequency (see U.S. Ser. No. 09/296,260, filed Jun. 6, 2001, in the name of Regeneron Pharmaceuticals, Inc., and incorporated by reference herein its entirety); and

(d) Use of this technology in combination with other existing methods that are used to regulate gene expression including, but not limited to, small molecule-regulated systems, recombinase-based systems, and regulated recombinase-based systems.

A preferred embodiment of the invention is a method of expressing a gene of interest in eukaryotic cells, comprising:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, and a gene of interest, and wherein the 5′ and 3′ homology arms are derived from a predetermined locus;

b) introducing the DNA targeting vector of (a) into eukaryotic cells such that the targeting vector integrates by homologous recombination into the predetermined locus; and

c) screening the eukaryotic cells of (b) to identify those cells in which the targeting vector has integrated by homologous recombination such that the targeted cells are capable of expressing the gene of interest.

Another preferred embodiment of the invention is a method of expressing a gene of interest in eukaryotic cells, comprising:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, and a gene of interest, and wherein the 5′ and 3′ homology arms are derived from an ubiquitously expressed locus;

b) introducing the DNA targeting vector of (a) into eukaryotic cells such that the targeting vector integrates by homologous recombination into the ubiquitously expressed locus; and

c) screening the eukaryotic cells of (b) to identify those cells in which the targeting vector has integrated by homologous recombination such that the targeted cells are capable of expressing the gene of interest.

Another preferred embodiment is a method of expressing a gene of interest in eukaryotic cells, comprising:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, and a gene of interest, and wherein the 5′ and 3′ homology arms are derived from the ROSA26 locus;

b) introducing the DNA targeting vector, of (a) into eukaryotic cells such that the targeting vector integrates by homologous recombination into the ROSA26 locus; and

c) screening the eukaryotic cells of (b) to identify those cells in which the targeting vector has integrated by homologous recombination such that the targeted cells are capable of expressing the gene of interest.

Also preferred is a method of expressing a gene of interest in embryonic stem cells, comprising:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, and a gene of interest, and wherein the 5′ and 3′ homology arms are derived from the ROSA26 locus;

b) introducing the DNA targeting vector of (a) into embryonic stem cells such that the targeting vector integrates by homologous recombination into the ROSA26 locus; and

c) screening the embryonic stem cells of (b) to identify those cells in which the targeting vector has integrated by homologous recombination such that the targeted cells are capable of expressing the gene of interest.

Also preferred is a method of expressing a gene of interest in embryonic stem cells, comprising:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, and a gene of interest, and wherein the 5′ and 3′ homology arms are derived from an ubiquitously expressed locus;

b) introducing the DNA targeting vector of (a) into embryonic stem cells such that the targeting vector integrates by homologous recombination into the ubiquitously expressed locus; and

c) screening the embryonic stem cells of (b) to identify those cells in which the targeting vector has integrated by homologous recombination such that the targeted cells are capable of expressing the gene of interest.

Yet another preferred embodiment is a method of genetically modifying a eukaryotic cell by integrating a nucleotide sequence into a predetermined locus, comprising:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, and the nucleotide sequence, and wherein the 5′ and 3′ homology arms are derived from the predetermined locus;

b) introducing the DNA targeting vector of (a) into eukaryotic cells such that the targeting vector integrates by homologous recombination into the predetermined locus; and

c) screening the eukaryotic cells of (b) to identify those cells that have been genetically modified by integrating a nucleotide sequence into a predetermined locus.

Yet another preferred embodiment is a method of genetically modifying a eukaryotic cell by integrating a nucleotide sequence into an ubiquitously expressed locus, comprising:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, and the nucleotide sequence, and wherein the 5′ and 3′ homology arms are derived from the ubiquitously expressed locus;

b) introducing the DNA targeting vector of (a) into eukaryotic cells such that the targeting vector integrates by homologous recombination into the ubiquitously expressed locus; and

c) screening the eukaryotic cells of (b) to identify those cells that have been genetically modified by integrating a nucleotide sequence into an ubiquitously expressed locus.

A preferred embodiment is a method of genetically modifying a eukaryotic cell by integrating a nucleotide sequence into the ROSA26 locus, comprising:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, and the nucleotide sequence, and wherein the 5′ and 3′ homology arms are derived from the ROSA26 locus;

b) introducing the DNA targeting vector of (a) into eukaryotic cells such that the targeting vector integrates by homologous recombination into the ROSA26 locus; and

c) screening the eukaryotic cells of (b) to identify those cells that have been genetically modified by integrating a nucleotide sequence into the ROSA26 locus.

Also preferred is a method of genetically modifying embryonic stem cells by integrating a nucleotide sequence into the ROSA26 locus, comprising:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, and the nucleotide sequence, and wherein the 5′ and 3′ homology arms are derived from the ROSA26 locus;

b) introducing the DNA targeting vector of (a) into embryonic stem cells such that the targeting vector integrates by homologous recombination into the ROSA26 locus; and

c) screening the embryonic stem cells of (b) to identify those cells that have been genetically modified by integrating a nucleotide sequence into the ROSA26 locus.

Also preferred is a method of genetically modifying embryonic stem cells by integrating a nucleotide sequence into an ubiquitously expressed locus, comprising:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, and the nucleotide sequence, and wherein the 5′ and 3′ homology arms are derived from the ubiquitously expressed locus;

b) introducing the DNA targeting vector of (a) into embryonic stem cells such that the targeting vector integrates by homologous recombination into the ubiquitously expressed locus; and

c) screening the embryonic stem cells of (b) to identify those cells that have been genetically modified by integrating a nucleotide sequence into an ubiquitously expressed locus.

Another preferred embodiment is a method of integrating a nucleotide sequence into a predetermined locus in eukaryotic cells, comprising:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, and the nucleotide sequence, and wherein the 5′ and 3′ homology arms are derived from the predetermined locus;

b) introducing the DNA targeting vector of (a) into eukaryotic cells such that the targeting vector integrates by homologous recombination into the predetermined locus; and

c) screening the eukaryotic cells of (b) to identify those cells in which the nucleotide sequence has integrated into the predetermined locus.

Another preferred embodiment is a method of integrating a nucleotide sequence into an ubiquitously expressed locus in eukaryotic cells, comprising:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, and the nucleotide sequence, and wherein the 5′ and 3′ homology arms are derived from the ubiquitously expressed locus;

b) introducing the DNA targeting vector of (a) into eukaryotic cells such that the targeting vector integrates by homologous recombination into the ubiquitously expressed locus; and

c) screening the eukaryotic cells of (b) to identify those cells in which the nucleotide sequence has integrated into the ubiquitously expressed locus.

Still another preferred embodiment is a method of integrating a nucleotide sequence into the ROSA26 locus in eukaryotic cells, comprising:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, and the nucleotide sequence, and wherein the 5′ and 3′ homology arms are derived from the ROSA26 locus;

b) introducing the DNA targeting vector of (a) into eukaryotic cells such that the targeting vector integrates by homologous recombination into the ROSA26 locus; and

c) screening the eukaryotic cells of (b) to identify those cells in which the nucleotide sequence has integrated into the ROSA26 locus.

Another preferred embodiment is a method of integrating a nucleotide sequence into the ROSA26 locus in embryonic stem cells, comprising:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, and the nucleotide sequence, and wherein the 5′ and 3′ homology arms are derived from the ROSA26 locus;

b) introducing the DNA targeting vector of (a) into embryonic stem cells such that the targeting vector integrates by homologous recombination into the ROSA26 locus; and

c) screening the embryonic stem cells of (b) to identify those cells in which the nucleotide sequence has integrated into the ROSA26 locus.

Another preferred embodiment is a method of integrating a nucleotide sequence into an ubiquitously expressed locus in embryonic stem cells, comprising:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, and the nucleotide sequence, and wherein the 5′ and 3′ homology arms are derived from the ubiquitously expressed locus;

b) introducing the DNA targeting vector of (a) into embryonic stem cells such that the targeting vector integrates by homologous recombination into the ubiquitously expressed locus; and

c) screening the embryonic stem cells of (b) to identify those cells in which the nucleotide sequence has integrated into the ubiquitously expressed locus.

Also preferred is a method of evaluating a gene product's biological activity, comprising:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, and a gene encoding the gene product, and wherein the 5′ and 3′ homology arms are derived from a predetermined locus;

b) introducing the DNA targeting vector of (a) into eukaryotic cells such that the targeting vector integrates by homologous recombination into the predetermined locus;

c) screening the eukaryotic cells of (b) to identify those cells in which the targeting vector has integrated by homologous recombination such that the targeted cells are capable of expressing the gene of interest; and

d) evaluating the gene product's biological activity.

Also preferred is a method of evaluating a gene product's biological activity, comprising:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, and a gene encoding the gene product, and wherein the 5′ and 3′ homology arms are derived from an ubiquitously expressed locus;

b) introducing the DNA targeting vector of (a) into eukaryotic cells such that the targeting vector integrates by homologous recombination into the ubiquitously expressed locus;

c) screening the eukaryotic cells of (b) to identify those cells in which the targeting vector has integrated by homologous recombination such that the targeted cells are capable of expressing the gene of interest; and

d) evaluating the gene product's biological activity.

Yet another preferred embodiment is a method of evaluating a gene product's biological activity, comprising:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, and a gene encoding the gene product, and wherein the 5′ and 3′ homology arms are derived from the ROSA26 locus;

b) introducing the DNA targeting vector of (a) into eukaryotic cells such that the targeting vector integrates by homologous recombination into the ROSA26 locus;

c) screening the eukaryotic cells of (b) to identify those cells in which the targeting vector has integrated by homologous recombination such that the targeted cells are capable of expressing the gene of interest; and

d) evaluating the gene product's biological activity.

Also preferred is a method of evaluating a gene product's biological activity, comprising:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, and a gene encoding the gene product, and wherein the 5′ and 3′ homology arms are derived from the ROSA26 locus;

b) introducing the DNA targeting vector of (a) into embryonic stem cells such that the targeting vector integrates by homologous recombination into the ROSA26 locus;

c) screening the embryonic stem cells of (b) to identify those cells in which the targeting vector has integrated by homologous recombination such that the targeted cells are capable of expressing the gene of interest; and

d) evaluating the gene product's biological activity.

Also preferred is a method of evaluating a gene product's biological activity, comprising:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, and a gene encoding the gene product, and wherein the 5′ and 3′ homology arms are derived from an ubiquitously expressed locus;

b) introducing the DNA targeting vector of (a) into embryonic stem cells such that the targeting vector integrates by homologous recombination into the ubiquitously expressed locus;

c) screening the embryonic stem cells of (b) to identify those cells in which the targeting vector has integrated by homologous recombination such that the targeted cells are capable of expressing the gene of interest; and

d) evaluating the gene product's biological activity.

Yet another preferred embodiment is a method of evaluating tissue-specific promoter activity, comprising:

a) constructing a DNA targeting vector, comprising:

a 5′ homology arm,

a protected transgene cassette, and

a 3′ homology arm,

wherein the protected transgene cassette is comprised of a transcriptional stop signal, a tissue-specific promoter, and a marker gene, and wherein the 5′ and 3′ homology arms are derived from a predetermined locus;

b) introducing the DNA targeting vector of (a) into eukaryotic cells such that the targeting vector integrates by homologous recombination into the predetermined locus;

c) screening the eukaryotic cells of (b) to identify those cells in which the targeting vector has integrated by homologous recombination such that the targeted cells are capable of expressing the marker gene; and

d) evaluating the tissue-specific promoter activity by observing the expression pattern of the marker gene.

Yet another preferred embodiment is a method of evaluating tissue-specific promoter activity, comprising:

a) constructing a DNA targeting vector, comprising:

a 5′ homology arm,

a protected transgene cassette, and

a 3′ homology arm,

wherein the protected transgene cassette is comprised of a transcriptional stop signal, a tissue-specific promoter, and a marker gene, and wherein the 5′ and 3′ homology arms are derived from an ubiquitously expressed locus;

b) introducing the DNA targeting vector of (a) into eukaryotic cells such that the targeting vector integrates by homologous recombination into the ubiquitously expressed locus;

c) screening the eukaryotic cells of (b) to identify those cells in which the targeting vector has integrated by homologous recombination such that the targeted cells are capable of expressing the marker gene; and

d) evaluating the tissue-specific promoter activity by observing the expression pattern of the marker gene.

Also preferred is a method of evaluating tissue-specific promoter activity, comprising:

a) constructing a DNA targeting vector, comprising:

a 5′ homology arm,

a protected transgene cassette, and

a 3′ homology arm,

wherein the protected transgene cassette is comprised of a transcriptional stop signal, a tissue-specific promoter, and a marker gene, and wherein the 5′ and 3′ homology arms are derived from the ROSA26 locus;

b) introducing the DNA targeting vector of (a) into eukaryotic cells such that the targeting vector integrates by homologous recombination into the ROSA26 locus;

c) screening the eukaryotic cells of (b) to identify those cells in which the targeting vector has integrated by homologous recombination such that the targeted cells are capable of expressing the marker gene; and

d) evaluating the tissue-specific promoter activity by observing the expression pattern of the marker gene.

Yet another preferred embodiment is a method of evaluating tissue-specific promoter activity, comprising:

a) constructing a DNA targeting vector, comprising:

a 5′ homology arm,

a protected transgene cassette, and

a 3′ homology arm,

wherein the protected transgene cassette is comprised of a transcriptional stop signal, a tissue-specific promoter, and a marker gene, and wherein the 5′ and 3′ homology arms are derived from the ROSA26 locus;

b) introducing the DNA targeting vector of (a) into embryonic stem cells such that the targeting vector integrates by homologous recombination into the ROSA26 locus;

c) screening the embryonic stem cells of (b) to identify those cells in which the targeting vector has integrated by homologous recombination such that the targeted cells are capable of expressing the marker gene; and

d) evaluating the tissue-specific promoter activity by observing the expression pattern of the marker gene.

Yet another preferred embodiment is a method of evaluating tissue-specific promoter activity, comprising:

a) constructing a DNA targeting vector, comprising:

a 5′ homology arm,

a protected transgene cassette, and

a 3′ homology arm,

wherein the protected transgene cassette is comprised of a transcriptional stop signal, a tissue-specific promoter, and a marker gene, and wherein the 5′ and 3′ homology arms are derived from an ubiquitously expressed locus;

b) introducing the DNA targeting vector of (a) into embryonic stem cells such that the targeting vector integrates by homologous recombination into the ubiquitously expressed locus;

c) screening the embryonic stem cells of (b) to identify those cells in which the targeting vector has integrated by homologous recombination such that the targeted cells are capable of expressing the marker gene; and

d) evaluating the tissue-specific promoter activity by observing the expression pattern of the marker gene.

An additional preferred embodiment is a method of evaluating the activity of the regulatory regions of a gene of interest, comprising:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, the regulatory regions to be evaluated, and a marker gene, and wherein the 5′ and 3′ homology arms are derived from a predetermined locus;

b) introducing the DNA targeting vector of (a) into eukaryotic cells such that the targeting vector integrates by homologous recombination into the predetermined locus;

c) screening the eukaryotic cells of (b) to identify those cells in which the targeting vector has integrated by homologous recombination such that the targeted cells are capable of expressing the marker gene; and

d) evaluating the activity of the regulatory regions of a gene of interest by observing the expression pattern of the marker gene.

An additional preferred embodiment is a method of evaluating the activity of the regulatory regions of a gene of interest, comprising:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, the regulatory regions to be evaluated, and a marker gene, and wherein the 5′ and 3′ homology arms are derived from an ubiquitously expressed locus;

b) introducing the DNA targeting vector of (a) into eukaryotic cells such that the targeting vector integrates by homologous recombination into the ubiquitously expressed locus;

c) screening the eukaryotic cells of (b) to identify those cells in which the targeting vector has integrated by homologous recombination such that the targeted cells are capable of expressing the marker gene; and

d) evaluating the activity of the regulatory regions of a gene of interest by observing the expression pattern of the marker gene.

Still yet another preferred embodiment is a method of evaluating the activity of the regulatory regions of a gene of interest, comprising:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, the regulatory regions to be evaluated, and a marker gene, and wherein the 5′ and 3′ homology arms are derived from ROSA26 locus;

b) introducing the DNA targeting vector of (a) into eukaryotic cells such that the targeting vector integrates by homologous recombination into the ROSA26 locus;

c) screening the eukaryotic cells of (b) to identify those cells in which the targeting vector has integrated by homologous recombination such that the targeted cells are capable of expressing the marker gene; and

d) evaluating the activity of the regulatory regions of a gene of interest by observing the expression pattern of the marker gene.

Also preferred is a method of evaluating the activity of the regulatory regions of a gene of interest, comprising:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, the regulatory regions to be evaluated, and a marker gene, and wherein the 5′ and 3′ homology arms are derived from the ROSA26 locus;

b) introducing the DNA targeting vector of (a) into embryonic stem cells such that the targeting vector integrates by homologous recombination into the ROSA26 locus;

c) screening the embryonic stem cells of (b) to identify those cells in which the targeting vector has integrated by homologous recombination such that the targeted cells are capable of expressing the marker gene; and

d) evaluating the activity of the regulatory regions of a gene of interest by observing the expression pattern of the marker gene.

Also preferred is a method of evaluating the activity of the regulatory regions of a gene of interest, comprising:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, the regulatory regions to be evaluated, and a marker gene, and wherein the 5′ and 3′ homology arms are derived from an ubiquitously expressed locus;

b) introducing the DNA targeting vector of (a) into embryonic stem cells such that the targeting vector integrates by homologous recombination into the ubiquitously expressed locus;

c) screening the embryonic stem cells of (b) to identify those cells in which the targeting vector has integrated by homologous recombination such that the targeted cells are capable of expressing the marker gene; and

d) evaluating the activity of the regulatory regions of a gene of interest by observing the expression pattern of the marker gene.

Also preferred is a non-human organism containing a genetically modified predetermined locus, wherein the modification is the introduction by homologous recombination into the predetermined locus a nucleotide sequence, comprising:

a 5′ homology arm,

a protected transgene cassette, and

a 3′ homology arm,

wherein the protected transgene cassette is comprised of a transcriptional stop signal, an exogenous promoter, and a gene of interest, and wherein the 5′ and 3′ homology arms are derived from the predetermined locus.

Still another preferred embodiment is a non-human organism wherein the predetermined locus is an ubiquitously expressed locus, including the ROSA26 locus.

Also preferred is a non-human organism which is a mouse.

An additional preferred embodiment is a non-human organism containing a genetically modified ubiquitously expressed locus, produced by a method comprising the steps of:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, and a gene of interest, and wherein the 5′ and 3′ homology arms are derived from an ubiquitously expressed locus;

b) introducing the DNA targeting vector of (a) into eukaryotic cells such that the targeting vector integrates by homologous recombination into the ubiquitously expressed locus;

c) screening the eukaryotic cells of (b) to identify those cells in which the targeted vector has integrated by homologous recombination;

d) introducing the eukaryotic cells of (c) into a blastocyst; and

e) introducing the blastocyst of (d) into a surrogate mother for gestation of the non-human organism containing the genetically modified ubiquitously expressed locus.

Also preferred is a non-human organism containing a genetically modified ROSA26 locus, produced by a method comprising the steps of:

a) constructing a DNA targeting vector, comprising:

a 5′ homology arm,

a protected transgene cassette, and

a 3′ homology arm,

wherein the protected transgene cassette is comprised of a transcriptional stop signal, an exogenous promoter, and a gene of interest, and wherein the 5′ and 3′ homology arms are derived from the ROSA26 locus;

b) introducing the DNA targeting vector of (a) into eukaryotic cells such that the targeting vector integrates by homologous recombination into the ROSA26 locus;

c) screening the eukaryotic cells of (b) to identify those cells in which the targeted vector has integrated by homologous recombination into the ROSA26 locus;

d) introducing the eukaryotic cells of (c) into a blastocyst; and

e) introducing the blastocyst of (d) into a surrogate mother for gestation of the non-human organism containing the genetically modified ROSA26 locus.

Yet another preferred embodiments is a non-human organism containing a genetically modified ROSA26 locus, produced by a method comprising the steps of:

a) constructing a DNA targeting vector, containing a nucleotide sequence, comprising:

a 5′ homology arm,

a protected transgene cassette, and

a 3′ homology arm,

wherein the protected transgene cassette is comprised of a transcriptional stop signal, an exogenous promoter, and a gene of interest, and wherein the 5′ and 3′ homology arms are derived from the ROSA26 locus;

b) introducing the DNA targeting vector of (a) into embryonic stem cells such that the targeting vector integrates by homologous recombination into the ROSA26 locus;

c) screening the eukaryotic cells of (b) to identify those cells in which the targeted vector has integrated by homologous recombination into the ROSA26 locus;

d) introducing the embryonic stem cells of (c) into a blastocyst; and

e) introducing the blastocyst of (d) into a surrogate mother for gestation of the non-human organism containing the genetically modified ROSA26 locus.

Yet another preferred embodiments is a non-human organism containing a genetically modified ubiquitously expressed locus, produced by a method comprising the steps of:

a) constructing a DNA targeting vector, containing a nucleotide sequence, comprising:

a 5′ homology arm,

a protected transgene cassette, and

a 3′ homology arm,

wherein the protected transgene cassette is comprised of a transcriptional stop signal, an exogenous promoter, and a gene of interest, and wherein the 5′ and 3′ homology arms are derived from an ubiquitously expressed locus;

b) introducing the DNA targeting vector of (a) into embryonic stem cells such that the targeting vector integrates by homologous recombination into the ubiquitously expressed locus;

c) screening the eukaryotic cells of (b) to identify those cells in which the targeted vector has integrated by homologous recombination into the ubiquitously expressed locus;

d) introducing the embryonic stem cells of (c) into a blastocyst; and

e) introducing the blastocyst of (d) into a surrogate mother for gestation of the non-human organism containing the genetically modified ubiquitously expressed locus.

Additional preferred embodiments of the methods of the invention further comprise neomycin, hygromycin, or puromycin; additional transcriptional stop signal sequences; regulatory elements, enhancers, silencers, or insulators; accessory elements, loxp sites, FRT sites, internal ribosome entry sites (IRES), or operators; recombinases including but not limited to Cre and FLP or FLPerecombinases, repressors including but not limited to the Tetracycline Repressor (TetR), transactivators including but not limited to the Tetracycline Transactivator (tTA); or lacZ, placental alkaline phosphatase, or any member of the fluorescent protein family.

Also preferred are embodiments wherein the embryonic stem cell is a mouse, rat, or other rodent embryonic stem cell; wherein the other embryonic stem cell is a chicken, rabbit, dog, cat, cow, horse, pig, sheep, or non-primate embryonic stem cell.

Also preferred is a DNA targeting vector, comprising:

a 5′ homology arm,

a protected transgene cassette, and

a 3′ homology arm,

wherein the protected transgene cassette is comprised of a transcriptional stop signal, an exogenous promoter, and a gene of interest, and wherein the 5′ and 3′ homology arms are derived from a predetermined locus.

Also preferred is a DNA targeting vector, comprising:

a 5′ homology arm,

a protected transgene cassette, and

a 3′ homology arm,

wherein the protected transgene cassette is comprised of a transcriptional stop signal, an exogenous promoter, and a gene of interest, and wherein the 5′ and 3′ homology arms are derived from an ubiquitously expressed locus.

Another preferred embodiment is a DNA targeting vector, comprising:

a 5′ homology arm,

a protected transgene cassette, and

a 3′ homology arm,

wherein the protected transgene cassette is comprised of a transcriptional stop signal, an exogenous promoter, and a gene of interest, and wherein the 5′ and 3′ homology arms are derived from the ROSA26 locus.

Other preferred embodiments are DNA targeting vectors further comprising neomycin, hygromycin, or puromycin; additional transcriptional stop signal sequences; regulatory elements, enhancers, silencers, or insulators; accessory elements, loxP sites, FRT sites, internal ribosome entry sites (IRES), or operators; recombinases including but not limited to Cre and FLPrecombinases, repressors including but not limited to the Tetracycline Repressor (TetR), transactivators including but not limited to the Tetracycline Transactivator (tTA); or lacZ, placental alkaline phosphatase, or any member of the fluorescent protein family.

Also preferred is a cell containing the DNA targeting vectors; the use of the cell to create non-human organisms, methods to evaluate a gene product's biological activity, methods to evaluate a tissue-specific promoter's activity, or methods to evaluate the activity of the regulatory regions of a gene of interest.

A preferred embodiment is one in which the blastocyst is a mouse, rat, or other rodent blastocyst; the surrogate mother is a mouse, rat, or other rodent.

Another preferred embodiment of the invention is one in which screening the eukaryotic cells comprises detecting the biological activity of the gene of interest.

Another preferred embodiment of the invention is one in which evaluating a gene product's biological activity comprises measuring its biological activity.

Another preferred embodiment of the invention is one in which evaluating the tissue-specific promoter activity comprises detecting expression of the gene of interest.

Another preferred embodiment of the invention is one in which evaluating the activity of the regulatory regions of a gene of interest comprises detecting the expression levels of the gene of interest.

A preferred embodiment of the invention is one in which the ubiquitously expressed locus is the BT-5 locus.

### Definitions

“Transgenic” cell or transgenic organism means a cell or organism that has been genetically altered so as to express a gene in a manner that is not normally expressed in that cell or organism.

“Promoter-less” means lacking a promoter that can confer expression in eukaryotic cells.

A “targeting vector” is a DNA vector that contains sequences “homologous” to endogenous chromosomal nucleic acid sequences flanking a desired genetic modification(s). The flanking homology sequences, referred to as “homology arms”, direct the targeting vector to a specific chromosomal location within the genome by virtue of the homology that exists between the homology arms and the corresponding endogenous sequence and introduce the desired genetic modification by a process referred to as “homologous recombination”.

“Homologous” means two or more nucleic acid sequences that are either identical or similar enough that they are able to hybridize to each other or undergo intermolecular exchange.

“Gene targeting” is the modification of an endogenous chromosomal locus by the insertion into, deletion of, or replacement of the endogenous sequence via homologous recombination using a targeting vector.

A “gene knock-out” is a genetic modification resulting from the disruption of the genetic information encoded in a chromosomal locus.

A “gene knock-in” is a genetic modification resulting from the replacement of the genetic information encoded in a chromosomal locus with a different DNA sequence.

A “knock-out organism” is an organism in which a significant proportion of the organism's cells harbor a gene knockout.

A “knock-in organism” is an organism in which a significant proportion of the organism's cells harbor a gene knock-in.

A “marker” or a “selectable marker” is a selection marker that allows for the isolation of rare transfected cells expressing the marker from the majority of treated cells in the population. Such marker's gene's include, but are not limited to, neomycin phosphotransferase and hygromycin B phosphotransferase, or fluorescing proteins such as GFP.

An “ES cell” is an embryonic stem cell. This cell is usually derived from the inner cell mass of a blastocyst-stage embryo.

An “ES cell clone” is a subpopulation of cells derived from a single cell of the ES cell population following introduction of DNA and subsequent selection.

A “flanking DNA” is a segment of DNA that is collinear with and adjacent to a particular point of reference.

A “non-human organism” is an organism that is not normally accepted by the public as being human.

“Orthologous” sequence refers to a sequence from one species that is the functional equivalent of that sequence in another species.

“Exogenous promoter” is a promoter that differs from the promoter(s) present in the targeted locus.

“Tissue-specific promoter” is a promoter that is expressed only in a subset of tissues or cell types in an organism.

“Ubiquitous promoter” is a promoter that is expressed in most cell types in the body.

An “ubiquitously expressed locus” is a locus that is expressed in most cell types in an organism.

A “predetermined locus” is a locus that has been successfully targeting by homologous recombination in eukaryotic cells.

A “protected transgene cassette” is a DNA sequence that minimally comprises a transcription termination signal, an exogenous promoter, and a transgene.

“Transgene cassette” is a DNA sequence containing a promoter, a gene of interest, a polyadenylation sequence and other regulatory or accessory elements.

The description and examples presented infra are provided to illustrate the subject invention. One of skill in the art will recognize that these examples are provided by way of illustration only and are not included for the purpose of limiting the invention.

### BRIEF DESCRIPTION OF THE FIGURES

FIG. 1:

An example of a conventional transgene cassette, consisting of a promoter, a transgene (usually the open reading frame of a gene), and a transcription termination signal (polyA). Note that other elements such as enhancers may be present as part of this cassette.

FIG. 2A:

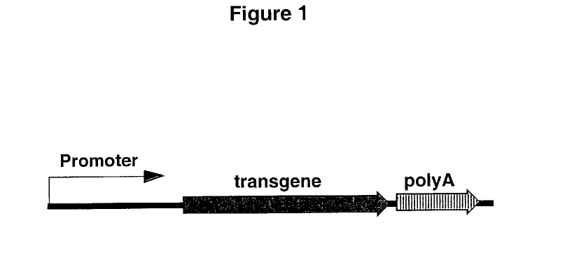

An example of a protected transgene cassette introduced downstream of the endogenous promoter of a targeted locus. The protected transgene cassette is shown integrated into the targeted locus. From 5′ to 3′, the cassette consists of:

(a) A transcription termination signal (polyA), which terminates transcription arising from the endogenous promoter (Pₑ) of the targeted locus,

(b) An exogenous promoter (Pₓ) driving the expression of a transgene, and

(c) A transcription termination signal (polyA), which terminates transcription arising from Pₓ

*Note that Pₑ is not part of the protected transgene cassette.

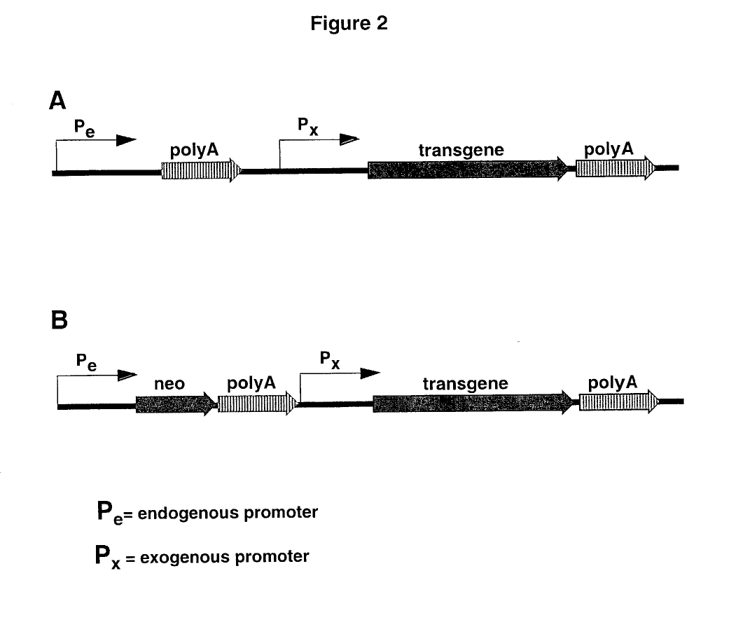

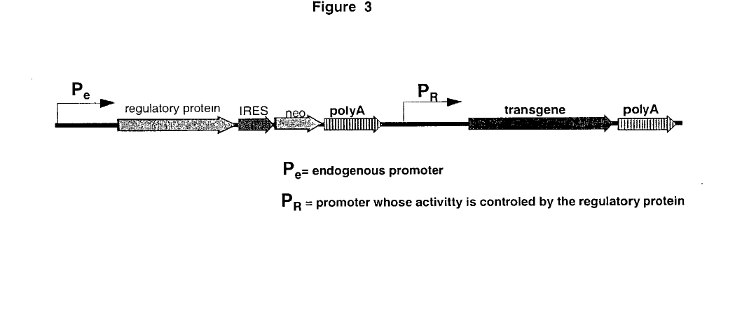

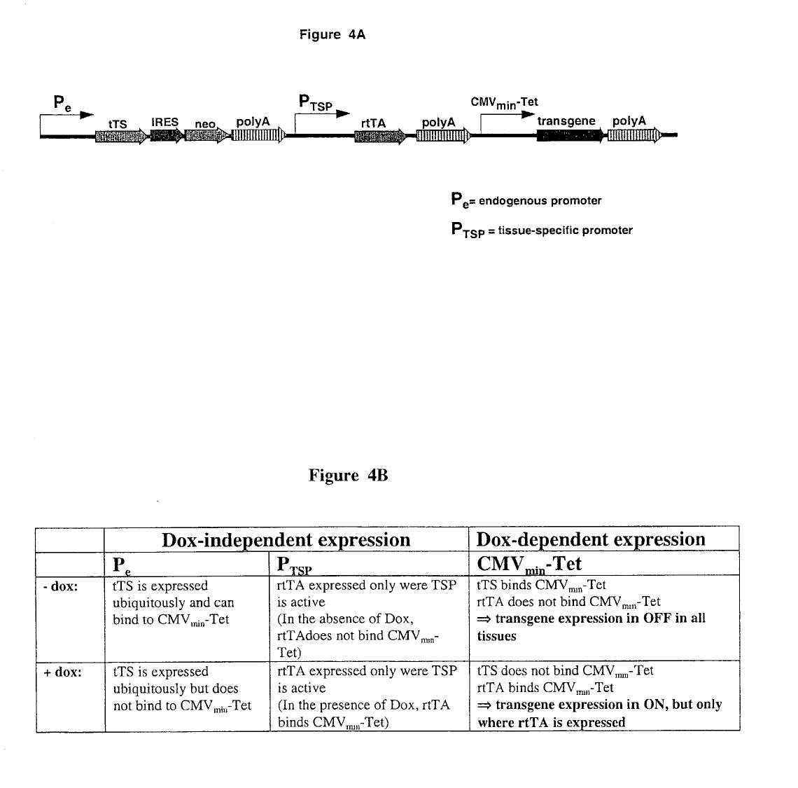

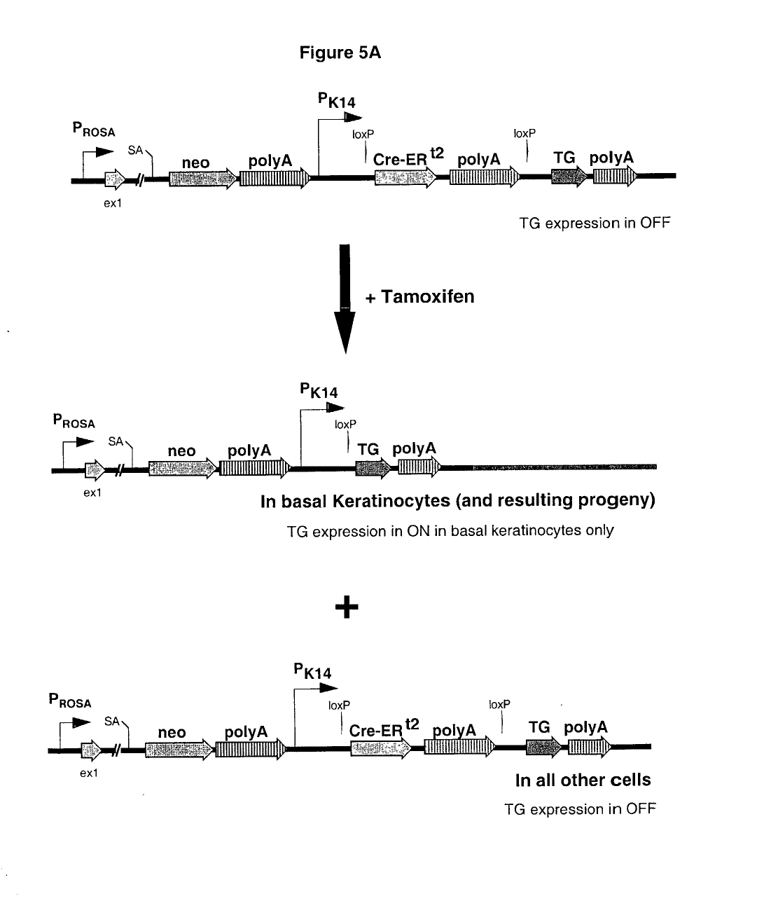

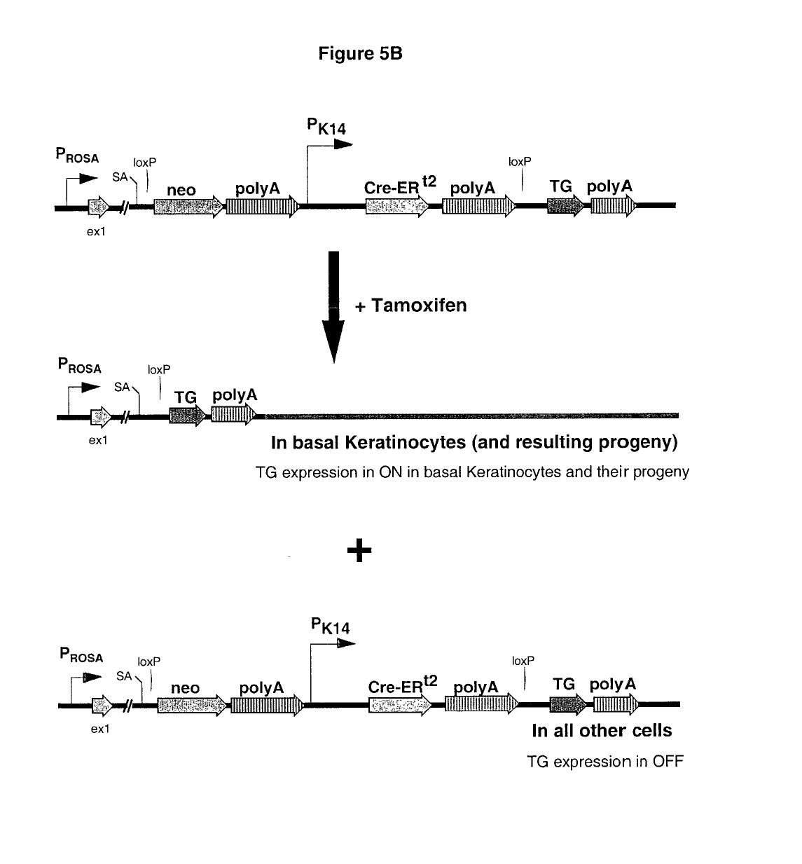

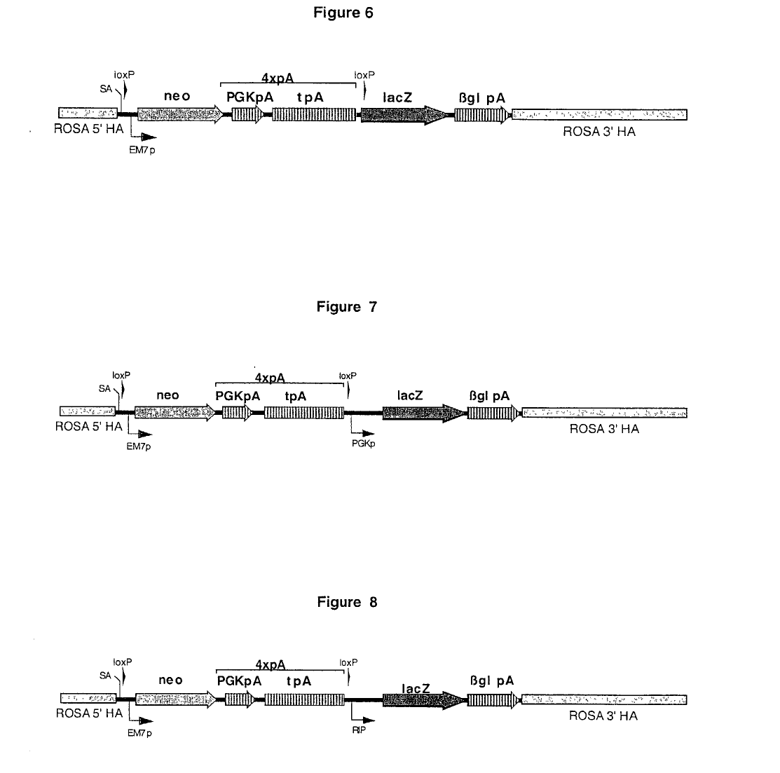

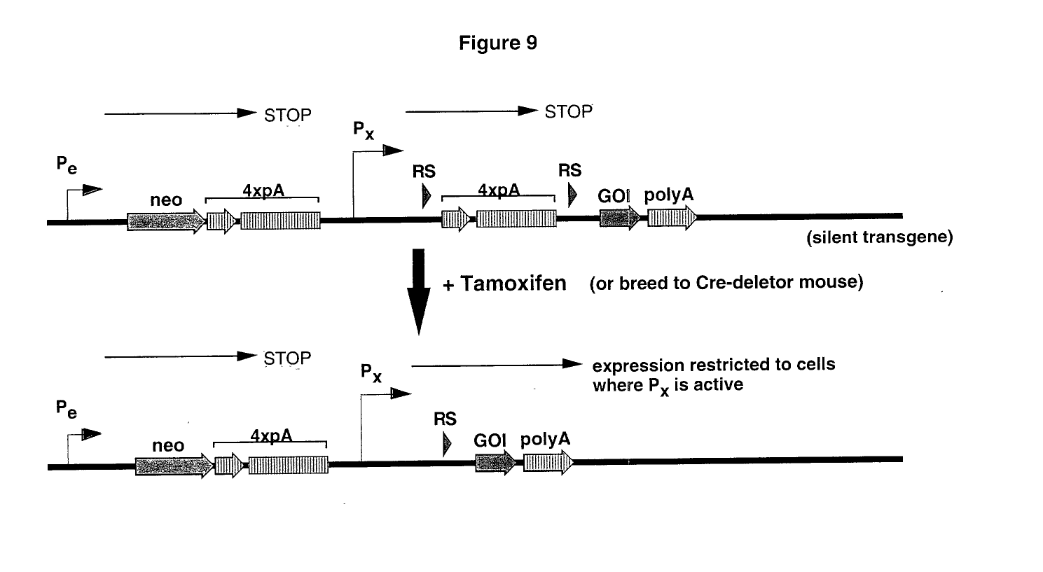

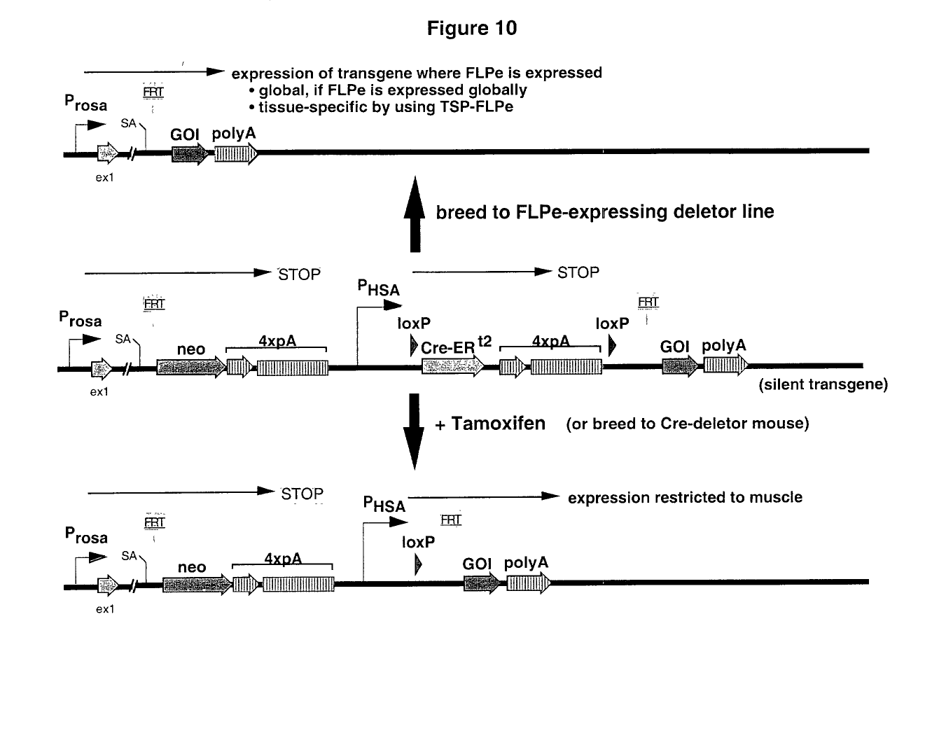

FIG. 2B:

An example of a protected transgene cassette introduced downstream of an endogenous promoter and utilizing a promoter-less drug selection gene. The protected transgene cassette is shown integrated into the targeted locus. From 5′ to 3′, the cassette consists of:

(a) A promoter-less drug selection marker, in this example neomycin phosphotransferase (neo), whose expression will be driven by the endogenous promoter (Pₑ) of the targeted locus,

(b) A transcription termination signal (polyA), which terminates transcription arising from the Pₑ,

(c) An exogenous promoter (Pₓ) driving the expression of a transgene, and

(d) A transcription termination signal (polyA), which terminates transcription arising from Pₓ

*Note that Pₑ is not part of the protected transgene cassette and that Pₑ must be active in the targeted cells, as expression of the drug selection gene is driven by that promoter.

FIG. 3:

A protected transgene cassette utilizing doxicycline-regulated gene expression technology. The protected transgene cassette is shown integrated into the targeted locus. From 5′ to 3′, the cassette consists of:

(a) A promoter-less small molecule-responsive transcription regulatory protein, such as the tetracycline-controlled transcriptional activator (tTA) or reverse tetracycline-controlled transcriptional activator (rtTA), followed by an internal ribosome entry site (IRES) and a drug selection marker, in this example neomycin phosphotransferase (neo), whose expression is driven by the endogenous promoter (Pₑ),

(b) A transcription termination signal (polyA), which terminates transcription arising from Pₑ,

(c) A regulated promoter (P_{R}) that is recognized by the small molecule-responsive transcription regulatory protein, and which drives expression of the transgene in a small-molecule-dependent fashion, and

(d) A transcription termination signal (polyA), which terminates transcription arising from P_{R}

*Note that Pₑ is not part of the protected transgene cassette and that Pₑ must be active in the targeted cells, as expression of the drug selection gene is driven by that promoter.

FIG. 4A:

A protected transgene cassette utilizing doxicycline-regulated gene expression technology employing both a transcriptional silencer and a transcriptional transactivator. The protected transgene cassette is shown integrated into the targeted locus. From 5′ to 3′, the cassette consists of:

(a) A promoter-less tetracycline-controlled transcriptional silencer (tTS), followed by an IRES and a drug selection marker, in this example neomycin phosphotransferase (neo), whose expression is driven by the endogenous promoter (Pₑ)

(b) A transcription termination signal (polyA), which terminate transcription arising from Pₑ,

(c) A tissue-specific promoter (P_{TSP}), driving the expression of the reverse tetracycline-controlled transcriptional activator (rtTA),

(d) A transcription termination signal (polyA), which terminates transcription arising from P_{TSP},

(e) The regulated promoter CMVₘᵢₙ-Tet, followed by a transgene, and

(f) A transcription termination signal (polyA), which terminates transcription arising from CMVₘᵢₙ-Tet

*Note that Pₑ must be active in the targeted cells, as expression of the drug selection gene is driven by that promoter.

FIG. 4B:

Schematic explanation of doxicycline-dependent control of gene expression as specified by the protected transgene cassette shown in FIG. 4A.

FIG. 5:

A protected transgene cassette utilizing tamoxifen-regulated gene expression technology employing the tamoxifen-inducible recombinase Cre-ER^{t2}. The protected transgene cassette is shown integrated into the targeted locus, using the ROSA26 locus as an example.

A. With loxP site flanking the Cre-ER^{t2}-polyA module of the cassette. From 5′ to 3′, the cassette consists of:

(a) A splice acceptor sequence,

(b) A promoter-less drug selection marker, in this example neomycin phosphotransferase (neo), whose expression will driven by the endogenous ROSA26 promoter (P_{ROSA}) after targeting,

(c) A transcription termination signal (polyA), which terminate transcription arising from P_{ROSA},

(d) A loxP site,

(e) A tissue-specific promoter (P_{K14}) which is expressed in basal keratinocytes, driving the expression of the tamoxifen-inducible recombinase Cre-ER^{t2},

(f) A transcription termination signal (polyA), which terminates transcription arising from P_{K14},

(g) A loxP site, placed in cis with the loxP site preceding Cre-ER^{t2}, in order to allow excision of the loxP-Cre-ER^{t2}-polyA-loxP module of the cassette in basal keratinocytes upon activation of Cre-ER^{t2} by tamoxifen,

(h) A transgene (TG), and

(i) A transcription termination signal (polyA), which terminates transcription arising from P_{K14} after excision of the loxP-Cre-ER^{t2}-polyA-loxP cassette.

B. With loxP site flanking the neo-polyA-P_{K14}-Cre-ER^{t2}-polyA module of the cassette. From 5′ to 3′, the cassette consists of:

(a) A splice acceptor sequence

(b) A lox P site,

(c) A promoter-less drug selection marker, in this example neomycin phosphotransferase (neo), whose expression will driven by the endogenous ROSA26 promoter (P_{ROSA}) after targeting,

(d) A transcription termination signal (polyA), which terminate transcription arising from P_{ROSA},

(e) A tissue-specific promoter (P_{K14}) which is expressed in basal keratinocytes, driving the expression of the tamoxifen-inducible recombinase Cre-ER^{t2},

(f) A transcription termination signal (polyA), which terminates transcription arising from P_{K14},

(g) A loxP site, placed in cis with the loxP site preceding Cre-ER^{t2}, in order to allow excision of the loxP-neo-polyA-P_{K14}-Cre-ER^{t2}-polyA-loxP module of the cassette in basal keratinocytes upon activation of Cre-ER^{t2} by tamoxifen,

(h) A transgene (TG), and

(i) A transcription termination signal (polyA), which terminates transcription arising from P_{K14} after excision of the loxP-Cre-ER^{t2}-polyA-loxP cassette.

The genomic structure of the targeted locus is shown pre- and post-addition of tamoxifen. Since the expression of Cre-ERt2 is limited to basal keratinocytes, excision of the floxed modules will occur only in that cell type and be present only in those cells and their progeny. (Floxed sequences or modules refers to sequences that are flanked by loxp sites). In all other cell types or cells of the epidermis that are descendants of basal keratinocytes and that have been exposed to tamoxifen, the targeted locus will remain unchanged. In A, post-tamoxifen, the transgene will be expressed only in basal keratinocytes, whereas in B, post-tamoxifen, the transgene will be expressed in basal keratinocytes and their descendants.

Note that neither exon of the ROSA26 locus nor the ROSA26 promoter (P_{ROSA}) are part of the protected transgene cassette used for targeting. P_{ROSA} is active in ES cells and, as a result, upon targeting, expression of the drug selection gene is driven by that promoter.

FIG. 6:

A schematic representation of a DNA targeting vector designed for targeting a protected transgene cassette (which, in this particular example, lacks an exogenous promoter) into the ROSA26 locus. The DNA targeting vector contains a 2.4 kb 5′ homology arm (ROSA 5′ HA) which contains sequence downstream of exon 1 of the ROSA26 locus; a promoter-less selection cassette containing SA-loxP-EM7-neo-4×polyA-loxP, wherein SA is a splice acceptor sequence, the two loxP sites are the locus of recombination sites derived from bacteriophage P1, the neomycin (neo) phosphotransferase gene, and 4×polyA which is a polyadenylation signal engineered by linking in tandem the polyadenylation signal of the mouse phosphoglycerate kinase gene (PGKpA) and three copies of a 254 bp BamHI fragment containing both early and late polyadenylation signals of Simian Virus 40 (tpA). This set of polyadenylation signals is referred to as 4×pA. The two loxP sites are in cis with respect to each other, in order to accommodate Cre-mediated excision rather than inversion of the floxed sequences. After the second loxP site, a LacZ open reading frame (ORF) has been engineered, followed by a rabbit β-globin polyA (βgl pA). The β-globin polyA is followed by a 3′ homology arm (ROSA 3′ HA) containing sequence continuous with the 5′ homology arm. The 3′ homology arm length is between 2 and 9.8 kb depending on the variant of the vector employed.

FIG. 7:

A schematic representation of a DNA targeting vector designed for targeting a protected transgene cassette into the ROSA26 locus. The DNA targeting vector shown here is identical to that shown in FIG. 6, except that the mouse phosphoglycerate kinase promoter (PGKp) has been included between the 2^{nd} loxP site and the LacZ ORF.

FIG. 8:

A schematic representation of a DNA targeting vector designed for targeting a protected transgene cassette into the ROSA26 locus. The DNA targeting vector shown here is identical to that shown in FIG. 6, except that the rat insulin promoter (RIP) has been included between the 2^{nd} loxP site and the LacZ ORF.

FIG. 9:

A protected transgene cassette requiring removal of a transcriptional stop sequence by a recombinase for expression of the transgene.

From 5′ to 3′, the cassette consists of:

(a) A promoter-less drug selection marker, in this example neomycin phosphotransferase (neo), whose expression is driven by the endogenous promoter (Pₑ),

(b) Transcription termination signals (4×polyA), which terminate transcription arising from Pₑ,

(c) An exogenous promoter (Pₓ), followed by a recombinase recognition site (RS),

(d) Transcription termination signals (4×polyA), which terminate transcription arising from P_{X,}, followed by another recombinase recognition site (RS),

(e) The transgene (GOI), and

(f) A transcription termination signal (polyA), which terminates transcription arising from Pₓ after deletion of the RS-polyA part of this cassette

*Notes:

(a) 4×polyA contains multiple polyadenylation signals. It has been engineered by linking in tandem the polyadenylation signal of the mouse phosphoglycerate kinase gene (PGKpA) and three copies of a 254 bp BamHI fragment containing both early and late polyadenylation signals of Simian Virus 40 (tpA), which are bidirectional. Together, this set of polyadenylation signals is referred to as 4×pA.

(b) Arrows followed by “STOP” indicate schematically the pre-mRNA transcribed from each cistron.

(c) As noted above, Pₑ must be active in the targeted cells, as expression of the drug selection gene is driven by that promoter.

FIG. 10:

A protected transgene cassette utilizing tamoxifen-regulated gene expression technology employing the tamoxifen-inducible recombinase Cre-ER^{t2}. The protected transgene cassette is shown integrated into the targeted locus, using the ROSA26 locus as an example. The transgene cassette employs a SA-FRT-EM7-neo-4×polyA selection mini-gene, wherein SA is a splice acceptor sequence, FRT is a site recognized by the FLP recombinase, the EM7 promoter is a bacterial promoter allowing for Kanamycin selection in E. Coli, the neomycin (neo) phosphotransferase gene, and 4×polyA which is a polyadenylation signal engineered by linking in tandem the polyadenylation signal of the mouse phosphoglycerate kinase gene (PGKpA) and three copies of a 254 bp BamHI fragment containing both early and late polyadenylation signals of Simian Virus 40 (tpA). A tissue-specific promoter derived from the human skeletal actin gene p_{HSA}, is placed after the 4×pA of the neo mini-gene. It is followed by a LoxP site, a CreER^{t2}-4×pA cassette, and then another LoxP and FRT site in tandem. After the 2^{nd} FRT site is the transgene (GOI) followed by another polyA. The two loxP sites and the two FRT sites are in cis with respect to each other, in order to accommodate Cre-mediated or FLP-mediated excision rather than inversion of the floxed or FRTed sequences respectively. Arrows followed by “STOP” indicate schematically the pre-mRNA transcribed from each cistron. Prior to addition of tamoxifen or introduction of Cre or FLP (by breeding to appropriate Cre or FLP deletor strains), the transgene is not expressed. PROSA drives expression of neo, and PHSA drives expression of CreER^{t2}.

After exposure to tamoxifen, the floxed CreER^{t2}-4×pA cassette is excised, but only in the cells where P_{HSA} is active, therefore allowing transcription of the transgene, restricted to the cells where P_{HSA} is active, thus achieving tissue-specific and tamoxifen-inducible expression of the transgene. Alternatively, the mice carrying this transgene cassette can be bred to Cre deletor mice that express Cre globally. In addition, in order to achieve transcription of the transgene by P_{ROSA} (which is expressed in the majority of cell types), the mice carrying the transgene cassette can be bred to a FLP deletor. This provides another level of flexibility and sophistication, as by breeding to a “global” FLP deletor such as ROSA26-FLPe, the FRT-neo-4×pA.PHSA-LoxP-CreERt2-4×pA.LoxP-FRT part of the cassette is deleted, leading to expression of the transgene where PROSA is active. An alternative strategy of employing a tissue-specific FPLe deletor is also attainable.

### DETAILED DESCRIPTION OF THE INVENTION

Currently available methods for generating transgenic animals include microinjection, including pronuclear injection, or using modified ES cells. It is generally desirable to be able to create transgenic animals in which the expression pattern of the transgene(s) is predicable. This is generally accomplished by carefully choosing a particular promoter whose activity is known. Also desirable are more complex situations wherein the promoter is accompanied by regulatory elements and/or accessory molecules that modulate its activity. As stated supra, it is always desirable to be able to generate transgenic animal lines whose phenotypes reflect transgene expression without the confounding complications of positional effects or insertional inactivation of endogenous loci.

Applicants, therefore, describe herein a new and novel method to express transgenes in vivo in a predictable and highly reproducible manner which also allows for spatial and temporal control of transgene expression. The method employs “protected transgene cassettes” that are targeted to predetermined loci, including ubiquitously expressed loci. This novel method allows the protected transgene cassettes to function as autonomous units that direct expression of the transgene(s) without being influenced by positional effects and where expression of the transgene is determined by the exogenous promoter and any optional regulatory or accessory elements present in the protected transgene cassette and not by the endogenous promoter of the targeted locus.

The protected transgene cassettes generally are minimally comprised of a transcription termination signal (polyA) followed by an exogenous promoter and a transgene representing a gene of interest. In addition, other regulatory and/or accessory elements may be present such as a drug selection genes, such as neomycin, hygromycin B, or puromycin; a polyadenylation signal (the polyA of the endogenous locus may also be employed); regulatory elements including, but not limited to, enhancers, silencers, and insulators; accessory elements such as loxp and FRT sites, internal ribosome entry sites (IRES), and operators such as the tetracycline operator; genes or cDNAs encoding for proteins that interact with these elements such as the Cre and FLP or FLPe recombinases and other related recombinases, the Tetracycline Repressor (TetR), the Tetracycline Transactivator (tTA), and others; or genes or cDNAs encoding for marker genes such as lacZ, placental alkaline phosphatase, or any member of the fluorescent protein family, or any other gene that can function as a marker gene. Insulators or chromatin boundary elements are specialized chromatin structures that regulate gene activity (Bell, et al., Science 291:447-450). Silencers are responsible for transcriptional repression in eukaryotes. There are two types, namely ‘silencer elements’ and ‘negative regulatory elements’ (NREs). Silencer elements are classical, position-independent elements that direct an active repression mechanism, and NREs are position-dependent elements that direct a passive repression mechanism (Ogbourne, S., et al., Biochem J Apr. 1, 1998;331 (Pt 1):1-14). In the protected transgene cassettes transcription from the endogenous promoter, which precedes the transcriptional stop signal, is blocked by the transcriptional stop signal, thus preventing the transcriptional machinery from reaching the exogenous promoter and the transgene (FIG. 2). This effectively “protects” the transgene from being transcribed by the endogenous promoter activity. Consequently, transgene expression is driven by the exogenous promoter only.

In order to ensure that the exogenous promoter also determines the expression pattern of the transgene, the protected transgene cassettes are introduced into predetermined loci, including ubiquitously expressed loci. Ubiquitously expressed loci are chosen because they are generally transcriptionally active in most cell types and, therefore, readily accessible to the transcriptional machinery. However, other loci are amenable to the methods of the invention such as predetermined loci, which are loci that have been successfully targeted by homologous recombination in eukaryotic cells, including ES cells. As a result, the method of the invention can preserve the expression pattern of an exogenous promoter, such that it mimics the expression pattern of this promoter in its native location in the genome. For example, if the exogenous promoter is a muscle-specific promoter engineered into the protected transgene cassette and then placed in the context of, for example, the ROSA26 locus, the activity of the promoter will occur in muscle. In addition, if a protected transgene cassette recapitulates experimentally favorable or useful aspects of the expression pattern of an exogenous promoter, then it can be useful for direct comparisons of multiple similarly configured transgene cassettes, where the transgene is varied and where the promoter and most other elements remain the same. It is relatively easy to validate the usefulness any given promoter by using the promoter to drive a marker gene such as LacZ, Green Fluorescent Protein (GFP) and its relatives or other fluorescent proteins, luciferase, or Placental Alkaline Phosphatase.

In accordance with the method of the invention, Applicants have created by way of non-limiting example, a protected transgene cassette that is targeted into an ubiquitously expressed locus, such as, for example, the ROSA26 locus (Zambrowicz et al., 1997, Proc Natl Acad Sci USA, 94, 3789-94) or the BT-5 locus (Michael et al., 1999, Mech Dev, 85, 35-47), although any predetermined locus, including other ubiquitously expressed loci may be suitable for use in the methods of the invention. Using this approach, the resulting expression pattern for the transgene is that specified by the exogenous promoter and any other optional associated regulatory/accessory elements present in the “protected transgene cassette”, examples of which are described supra. Any promoter that directs levels of expression different from the endogenous promoter of the targeted locus, or any tissue-specific promoter that directs expression only in a limited number of tissues or cell types, or any other regulated promoters such as those whose activity is controlled by small molecules like tetracycline, can be used in the methods of the invention. Non-limiting examples of tissue-specific promoters are shown in Table 1. This should enable the use of nearly any promoter and associated regulatory elements without their activities being subject to positional effects. Finally, the protected transgene cassette is inserted into the chosen chromosomal locus without making any major alterations in the endogenous locus so as to prevent any changes in the regulation or pattern of expression of the endogenous locus. This is a clear difference from and improvement over what has been done in the past (i.e. studies utilizing the hrpt locus).

In order to maintain fidelity of the expression pattern expected for the exogenous promoter of choice, Applicants realized it would be necessary to prevent transcription originating from the endogenous promoter of the targeted locus from accessing: (a) the exogenous promoter (to avoid promoter interference);(b) the transgene(s) (to avoid expression of the transgene from the endogenous promoter of the targeted locus); and (c) any accessory or regulatory elements that may be part of the protected transgene cassette. To achieve this, Applicants engineered the protected transgene cassettes such that the exogenous promoter is preceded by a transcription termination signal (FIG. 2A). When these protected transgene cassettes are introduced by homologous recombination into the endogenous chromosomal locus, transcription from the endogenous promoter cannot reach the exogenous promoter in the protected transgene cassette or access the transgene(s) and any accompanying regulatory elements. In addition, in an alternative embodiment, the endogenous promoter can be used to drive expression of a drug selection marker gene such as neomycin that is, in turn, followed by the transcription termination signal (FIG. 2B). This configuration can be particularly useful as it can serve the dual purpose of providing a drug selection and a termination of transcription from the endogenous promoter. Furthermore, as ubiquitously expressed chromosomal loci are nearly always also expressed in ES cells (Bronson et al., 1996, Proc Natl Acad Sci USA, 93, 9067-72.; Friedrich and Soriano, 1991, Genes Dev, 5, 1513-23.; Michael et al., 1999, Mech Dev, 85, 35-47.; Soriano, 1999, Nat Genet, 21, 70-1.; Zambrowicz et al., 1997, Proc Natl Acad Sci USA, 94, 3789-94), this configuration can be combined with previously developed methodology (see U.S. Ser. No. 09/296,260, filed Jun. 6, 2001, in the name of Regeneron Pharmaceuticals, Inc., and incorporated by reference herein its entirety) that provides a selection scheme wherein only correctly targeted ES cell clones are selected, therefore achieving nearly 100% targeting efficiency.

Finally, in order to ensure that the exogenous promoter and any associated regulatory/accessory elements will also determine the pattern of transgene expression and result in a transgene expression pattern that mimics the choice of promoter and any associated regulatory/accessory elements, the protected transgene cassettes are targeted into predetermined, including transcriptionally active, ubiquitously expressed loci, such as, but not limited to, the ROSA26 or the BT-5 loci. In addition to ensuring that the desired control over gene expression is achieved, targeting into these loci confers several other advantages:

(a) Avoiding positional effects on the expression pattern of the transgene.

(b) Ensuring a highly reproducible pattern of expression that is dependent on the choice of the exogenous promoter and any optional regulatory/accessory elements and which is transgene identity-independent.

(c) Modifying loci with well understood biological properties. Preferably loci which can be knocked out without any adverse phenotypes are used, so as to allow breeding transgenics lines to homozygocity or to allow crossbreeding of different lines to create animals with multiple transgenes. Both the ROSA26 and the BT-5 locus fulfill these criteria (Michael et al., 1999, Mech Dev, 85, 35-47.; Zambrowicz et al., 1997, Proc Natl Acad Sci USA, 94, 3789-94), although other loci are also suitable.

(d) Afford direct transgene to transgene comparisons, since the variables in the targeting vector can be minimized to just a single variable per targeting vector. For example, several mutant forms of a single gene can be compared without confounding issues such as variability of gene expression and positional effects associated with traditional transgenic animal technology, since the only parameter that differs between transgenic animal lines is the particular mutant form of the gene. In this manner even mutations that would lead to very subtle changes in phenotype can be compared in vivo.

(e) Afford comparison of promoter, promoter elements, and regulatory sequences in vivo. In this example, the exogenous promoter and/or regulatory elements are varied whereas the other elements of the protected transgene cassette and the targeted locus remain the same. Instead of a gene of interest, a marker gene such as LacZ, GFP or GFP relatives, or Placental Alkaline Phosphatase is used.

Based on the above general concept, several advanced embodiments of protected transgene cassettes have been engineered, some of which are described herein as non-limiting examples contemplated by the subject invention.

Protected Transgene Cassettes Utilizing Small Molecule-Regulated Gene Expression Technologies.

In this embodiment of the invention, the endogenous promoter is used to drive expression of regulatory proteins that, in turn, control transgene expression from an exogenous promoter whose activity is regulated by these regulatory proteins. Examples of suitable regulatory proteins are the tetracycline transactivator (tTA) (Gossen and Bujard, 1992, Proc Natl Acad Sci USA, 89, 5547-51), the reverse tetracycline transactivator (rtTA) (Gossen et al., 1995, Science, 268, 1766-9), the tetracycline repressor (TetR) (Altschmied and Hillen, 1984, Nucleic Acids Res, 12, 2171-80.; Blau and Rossi, 1999, Proc Natl Acad Sci USA, 96, 797-9.; Parge et al., 1984, J Mol Biol, 180, 1189-91), and the tetracycline-controlled transcriptional silencer (tTS) (Freundlieb et al., 1999, J Gene Med, 1, 4-12.; Witzgall et al., 1994, Proc Natl Acad Sci USA, 91, 4514-8), each of whose activity is regulated by small molecules such as tetracycline or tetracycline analogs and derivatives such as doxicycline (Baron and Bujard, 2000, Methods Enzymol, 327,401-21; Blau and Rossi, 1999, Proc Natl Acad Sci USA, 96, 797-9.; Freundlieb et al., 1999, J Gene Med, 1, 4-12.; Gossen and Bujard, 1992, Proc Natl Acad Sci USA, 89, 5547-51.; Kistner et al., 1996, Proc Natl Acad Sci USA, 93, 10933-8.; Shockett and Schatz, 1996, Proc Natl Acad Sci USA, 93, 5173-6). In this protected transgene cassette, the regulatory protein gene or cDNA is followed by a transcription termination signal(s). The exogenous promoter is a promoter that is regulated by the regulatory protein. For example, if the regulatory protein is tTA or rtTA or a combination of rtTA and tTS then the exogenous promoter can be the CMV minimal promoter with Tet operator sites (CMVₘᵢₙ-Tet) (FIG. 3). Usually, a drug selection marker gene is also included either following or preceding the regulatory protein gene but, in any event, before the transcription termination signal. An internal ribosome entry site (IRES) (Kozak, 2001, Mol Cell Biol, 21, 1899-907.; Martinez-Salas, 1999, Curr Opin Biotechnol, 10, 458-64) is placed between the two genes or cDNAs to assure that the second component of the resulting bicistronic message is translated efficiently. Thus, in this embodiment, the endogenous promoter is driving expression of the regulatory protein and the drug selection marker genes. The advantage of this embodiment is that it allows for precise switching (turning on and turning off) of the transgene. Note that other combinations of promoters and regulatory proteins and corresponding DNA elements can be designed to create additional specific embodiments of the method of the invention.

Another embodiment of the invention uses the endogenous promoter to drive expression of a transcriptional silencer gene and the exogenous promoter is used to drive transcription of the corresponding transactivator. A second exogenous promoter whose activity is controlled negatively by the transcriptional silencer and positively by the transcriptional regulator is used to drive expression of the transgene. In one example of this embodiment, the endogenous promoter drives expression of the tetracycline-controlled transcriptional silencer (tTS); since the endogenous promoter is an ubiquitous promoter, tTS is expressed in all tissues. The exogenous promoter, which in this embodiment is generally a tissue-specific promoter, drives expression of the tetracycline reverse transactivator (rtTA), whose expression is thereby restricted only to the tissues wherein the exogenous promoter is active. Finally, an additional exogenous promoter, for example the CMV minimal promoter with Tet operator sites (CMVₘᵢₙ-Tet), drives expression of the transgene (FIG. 4). The CMVₘᵢₙ-Tet promoter requires binding by the rtTA to initiate transcription. In the absence of doxicycline, rtTA does not bind to CMVₘᵢₙ-Tet and, therefore, transcription from this promoter is very low. In addition, in the absence of doxicycline, tTS binds to CMVₘᵢₙ-Tet and suppresses any residual or background transcription from this promoter. The advantage of expressing tTS ubiquitously is that it blocks residual or background expression from the CMVₘᵢₙ-Tet in all tissues, thus ensuring essentially undetectable levels of transgene expression in the absence of doxicycline. In addition, restricting the expression of the transactivator rtTA to only the tissues where expression of the transgene is desired provides one more level of stringency. Upon addition of doxicycline, tTS will stop binding to CMVₘᵢₙ-Tet. rtTA will now bind but only in the tissue(s) where it is expressed as directed by the tissue-specific promoter, thereby inducing transgene expression in those tissues only.

Protected Transgene Cassettes Utilizing Cre or FLP and Their Variants or Other Recombinases to Regulate Transgene Expression.

In this embodiment of the invention the endogenous promoter (Pₑ) is used to drive expression of a drug resistance marker such as neomycin phosphotransferase (neo) which is followed by poloyadenylation signals (4×pA), to ensure both efficient polyadenylation of the neo message and termination of transcription before the exogenous promoter. The reasoning behind using multiple, in this example four polyadenylation signals is to ensure that transcription of the neo gene will not extend into the gene driven by the exogenous promoter (Pₓ) Note however, that a single polyadenylation signal or other combinations of polyadenylation signals may be equally efficient. The exogenous promoter may be a tissue-specific promoter or any other promoter that expresses in mammalian cells. The exogenous promoter another 4×pA. This 4×pA is flanked by sites (RS) that recognized by recombinases that mediate excision of the sequences flanked by these sites. As already explained above, examples of pairs of such recombinases/recognition sites are Cre/LoxP (Abremski and Hoess, 1984, J Biol Chem, 259, 1509-14; Araki et al., 1997, J Biochem (Tokyo), 122, 977-82) and FLP/FRT (Andrews et al., 1985, Cell, 40, 795-803; Cox, 1983, Proc Natl Acad Sci USA, 80,4223-7; Meyer-Leon et al., 1984, Cold Spring Harb Symp Quant Biol, 49, 797-804), but variants of these recombinases such as mutated Cre and corresponding mutated Lox sites (Buchholz and Stewart, 2001, Nat Biotechnol, 19, 1047-52.; Shimshek et al., 2002, Genesis, 32, 19-26) or the improved FLP variant FLPe (Buchholz et al., 1998, Nat Biotechnol, 16, 657-62) or other recombinases/recognition sites may also be used (Grainge and Jayaram, 1999, Mol Microbiol, 33, 449-56). The LoxP or FRT sites are placed in cis with respect to each other. In this manner, the sequence between the sites—in this embodiment the 4×polyA—is excised upon encountering the recombinase. Thus expression of the transgene is dependent on the presence of the corresponding recombinase. One strategy to introduce the required recombinase is by breeding mice carrying the Pₑ-neo-4×polyA-Pₓ-RS-polyA-RS-transgene-polyA cassette with mice carrying the corresponding recombinase (that recognizes RS) expressed in ubiquitous fashion. The resulting double heterozygous progeny—i.e. progeny carrying both the engineered transgene locus and the recombinase gene—will have a rearranged transgene locus where the RS-polyA has been removed. Consequently the transgene will be expressed from the exogenous promoter Pₓ (FIG. 9). Note that a similar strategy can be employed using small molecule-regulated recombinases as described in other embodiments below.

Protected Transgene Cassettes Utilizing Small Molecule-Regulated Recombinases to Regulate Gene Expression

In this embodiment of the invention, the exogenous promoter drives the expression of regulated recombinases such as CreER (Schwenk et al., 1998, Nucleic Acids Res, 26, 1427-32; Vooijs et al., 2001, EMBO Rep, 2, 292-297) and its variants (Kellendonk et al., 1996, Nucleic Acids Res, 24, 1404-11) or FLP-LBD (Nichols et al., 1997, Mol Endocrinol, 11, 950-61), followed by a transcription termination signal. These recombinases are largely inactive in eukaryotic cells, because they are kept in an unfolded or inappropriately folded state by the fused estrogen receptor ligand binding domain, often referred to as ER of LBD. Addition of the cognate ligand, e.g. tamoxifen or 4-OH-tamoxifen for CreER^{t} (Vooijs et al., 2001, EMBO Rep, 2, 292-297) or CreER^{t2} (Indra et al., 1999, Nucleic Acids Res, 27, 4324-7.; Vallier et al., 2001, Proc Natl Acad Sci USA, 98, 2467-72), leads to activation of the recombinase activity and excision or inversion of the sequences flanked by the loxP sites (Abremski and Hoess, 1984, J Biol Chem, 259, 1509-14; Hamilton and Abremski, 1984, J Mol Biol, 178, 481-6). Whether inversion or excision takes place is a function of the direction of the loxP sites with respect to each other: if they are direct repeats, then excision takes place; if they are inverted with respect to each other, then inversion takes place (Joyner, 1999, The Practical Approach Series, 293). Inclusion of regulated recombinases allows for induction of transgene expression through removal of a ‘floxed’ or ‘FRTed’ transcription termination signal. A floxed sequence is a DNA sequence flanked by loxP sites, which are sites recognized by the Cre recombinase (Abremski and Hoess, 1984, J Biol Chem, 259, 1509-14; Araki et al., 1997, J Biochem (Tokyo), 122, 977-82) or its variants (Joyner, 1999, The Practical Approach Series, 293; Vooijs et al., 2001, EMBO Rep, 2, 292-297); and references within). A “FRTed” sequence is a DNA sequence flanked by FRTFRT sites, which are sites that are recognized by the FLP recombinase (Andrews et al., 1985, Cell, 40, 795-803; Cox, 1983, Proc Natl Acad Sci USA, 80, 4223-7; Meyer-Leon et al., 1984, Cold Spring Harb Symp Quant Biol, 49, 797-804) or its variants (Buchholz et al., 1998, Nat Biotechnol, 16, 657-62; Joyner, 1999, The Practical Approach Series, 293; Nichols et al., 1997, Mol Endocrinol, 11, 950-61). If these recombinases are going to be incorporated into the transgene cassette together with their cognate recombination sites, e.g. loxP and FRT for Cre and FLP, respectively, then it is advisable to engineer an intron within the recombinase gene in order to avoid rearrangement of the sequence flanked by the recombination sites while the cassette is being constructed in E. coli. Since eukaryotic introns are not processed in E. coli, intron-containing recombinases are inactive in E. coli (Bunting et al., 1999, Genes Dev, 13, 1524-8).

In one non-limiting example of this embodiment, the endogenous promoter is the ROSA26 promoter (P_{ROSA}) and is used to drive a drug selection marker gene such as neomycin (neo). Transcription from P_{ROSA} is terminated at a transcription termination signal placed after the neo gene. The exogenous promoter is a tissue-specific promoter, such as the keratin 14 promoter (P_{K14}) (Vassar et al., 1989, Proc Natl Acad Sci USA, 86, 1563-7), and drives expression of a regulated recombinase, such as CreER^{t2}, followed by a transcription termination signal, the transgene, and yet a third transcription termination signal. Prior to addition of 4-OH-tamoxifen, the endogenous promoter, P_{ROSA}, drives neo expression ubiquitously, whereas the P_{K14} drives expression of CreER^{t2} only in basal keratinocytes (Indra et al., 1999, Nucleic Acids Res, 27, 4324-7). If the CreER^{t2}-polyA component of the cassette is floxed in cis (i.e. flanked by loxP sites that are direct repeats with respect to each other), then upon addition of 4-OH-tamoxifen, the CreER^{t2}-polyA component of the cassette will self-excise (but only in the basal keratinocytes, because expression from P_{K14} is restricted to that cell type), and P_{K14} will now drive expression of the transgene (FIG. 5A). Note that expression of the transgene will be restricted to basal keratinocytes. Expression from the endogenous promoter, PROSA, will not be affected and will not contribute to the expression of the transgene as the latter is still ‘protected’ by the transcription termination signal downstream of neo.

If the neo-polyA-PK₁₄-CreER^{t2}-polyA component of the cassette is floxed in cis, then upon addition of 4-OH-tamoxifen, the neo-polyA-PK₁₄-CreER^{t2}-polyA component of the cassette will self-excise (but, as mentioned above, excision will be limited to basal keratinocytes) and, as a result, the endogenous promoter, P_{ROSA}, will drive expression of the transgene (FIG. 5B). Note that in this case expression of the transgene will be not be restricted to basal keratinocytes, but will also include their progeny including more differentiated cells of the epidermis, since P_{ROSA} is active in these cells. In cells where the excision reaction has not taken place, the P_{ROSA} will continue driving expression of neo, and the gene of interest will not be expressed in these cells.

Therefore, by choosing where to position the loxP sites, one can design the system to achieve subtly different expression profiles of the transgene while utilizing the same repertory of promoters and regulated recombinases.

In another non-limiting example of this embodiment, the endogenous promoter is the ROSA26 promoter (P_{ROSA}) and is used to drive a drug selection marker gene such as neo. A FRT site (recognized by the FLP recombinase) has been engineered between the splice acceptor (past exon 1 of the ROSA26 locus) and the neo ORF. Transcription from P_{ROSA} is terminated at transcription termination signals (4×pA—see also FIG. 6) placed after the neo gene. The reasoning behind using multiple, in this example four polyadenylation signals is to ensure that transcription of the ROSA26-neo hybrid gene will not extend into the gene driven by the exogenous promoter, in this example P_{HSA} (see infra). Note however, that a single polyadenylation signal or other combinations of polyadenylation signals may be equally efficient. The exogenous promoter is a tissue-specific promoter, in this example the human skeletal actin promoter (P_{HSA}) whose expression is largely restricted to muscle cells (Brennan and Hardeman, 1993, J Biol Chem, 268, 719-25). P_{HSA} drives expression of a regulated recombinase, in this example CreER^{t2}, followed by another 4×pA. The CreER^{t2}-4×pA part of this cassette is flanked by LoxP sites positioned in cis with respect to each other—for this example, the LoxP-CreER^{t2}-4×pA-LoxP cassette will be hereafter referred to as the floxed CreER^{t2} cassette. The floxed CreER^{t2} cassette is a ‘self-deleting’ cassette. Meaning that upon exposure to tamoxifen activation of CreER^{t2} will lead to excision of the sequence encoding it (and the accompanying 4×pA) as it is flanked by LoxP sites. After the 4×pA of the P_{HSA}-LoxP-CreER^{t2}-4×pA-LoxP cassette, a FRT site has been placed such that it is in the cis orientation with respect to the FRT site place in front of the neo ORF, and it is followed by the transgene (gene of interest), and yet a third transcription termination signal (FIG. 10). Prior to addition of 4-OH-tamoxifen, the endogenous promoter, P_{ROSA}, drives neo expression ubiquitously, whereas the P_{HSA} drives expression of CreER^{t2} primarily in muscle cells (Brennan and Hardeman, 1993, J Biol Chem, 268, 719-25). Since the CreER^{t2}-polyA component of the cassette is floxed in cis, upon addition of 4-OH-tamoxifen, the floxed CreER^{t2} cassette will self-excise (but only in the cells where P_{HSA} directs transcription). Consequently, by removing LoxP-CreER^{t2}-4×pA, the transgene will now be expressed from the HSA promoter in place of CreER^{t2}. Note that expression of the transgene will be restricted to the cells where P_{HSA} is active. Note also that between the P_{HSA} promoter and the transgene (GOI) remain a LoxP and a FRT site. Thus, if either the rearranged locus (after deletion of the floxed CreER^{t2} cassette by activation of CreER^{t2} with tamoxifen) or the original allele are subject to modification by FLP or FLPe. Exposure to FLPe will lead to excision of the part of the transgene cassette that is flanked by FRT sites. In this configuration of the transgene cassette this will result in removal of FRT-neo-4×pA.PHSA-LoxP-CreER^{t2}-4×pA-LoxP, leaving on the genome the FRT-transgene-polyA part of the cassette. Therefore excision of the FRTed cassette will lead to expression of the transgene from the endogenous promoter, in this example the ROSA26 promoter, thereby resulting in ubiquitous expression of the transgene (FIG. 10). An alternative possibility would be to breed the mice bearing this transgenic locus with FLPe deletor mice where expression of FLPe is restricted to only certain cell types, to achieve tissue specific expression of the transgene, other than that which can be afforded by P_{HSA} This type of transgenic locus configuration allows maximum flexibility of transgene expression with respect to tissue specificity and inducability, while minimizing the number of initial transgenic lines that have to be engineered as well as consequent breeding steps.

Therefore, one may take advantage of multiple recombinase/recognition site systems to design regulated expression systems to achieve multiple specificities in the obtainable expression patterns, depending on which recombinase and/or mouse breeding strategy is utilized.

The description and examples presented infra are provided to illustrate the subject invention. One of skill in the art will recognize that these examples are provided by way of illustration only and are not included for the purpose of limiting the invention.

### EXAMPLES

Many of the techniques used to construct the DNA vectors and the protected transgene cassettes described herein are standard molecular biology techniques well known to the skilled artisan (see e.g., Sambrook, J., E. F. Fritsch And T. Maniatis. Molecular Cloning: A Laboratory Manual, Second Edition, Vols 1, 2, and 3, 1989; Current Protocols in Molecular Biology, Eds. Ausubel et al., Greene Publ. Assoc., Wiley Interscience, NY). All DNA sequencing is done by standard techniques using an ABI 373A DNA sequencer and Taq Dideoxy Terminator Cycle Sequencing Kit (Applied Biosystems, Inc., Surrogate City, Calif.).

### Example 1

A DNA targeting vector was constructed consisting of an approximately 2.4 kb 5′ homology arm containing sequence downstream of exon 1 of the ROSA26 locus extending from the NotI site to the NheI site (Friedrich and Soriano, 1991, Genes Dev, 5, 1513-23.; Soriano, 1999, Nat Genet, 21, 70-1). A protected transgene cassette was inserted at that site. The protected transgene cassette in this example is SA-loxP-EM7-neo-4×polyA-loxP-lacZ-β-globin polyA, wherein SA is a splice acceptor sequence, the two loxP sites are the locus of recombination sites derived from bacteriophage P1 (Abremski and Hoess, 1984, J Biol Chem, 259, 1509-14), EM7 is a prokaryotic constitutively active promoter, neo is the neomycin phosphotransferase gene (Beck et al., 1982, Gene, 19, 327-36), and 4×polyA is a polyadenylation signal engineered by linking in tandem the polyadenylation signal of the murine pgk gene (Adra et al., 1987, Gene, 60, 65-74) and three copies of a 254 bp BamHI fragment containing both early and late polyadenylation signals of Simian Virus 40 (SV40) (Reddy et al., 1978, Science, 200, 494-502; Thimmappaya et al., 1978, J Biol Chem, 253, 1613-8), lacZ is an open reading frame (ORF) encoding for E. coli β-galactosidase, and β-globin polyA is a polyadenylation signal derived from the rabbit β-globin gene. The β-globin polyA is followed by a 3′ homology arm containing sequence continuous to that of the 5′ homology arm. The 3′ homology arm extends approximately 9.8 kb past the site of insertion of the protected transgene cassette and contains ROSA26 sequence up to the unique EcoRI site (FIG. 6). Note the absence of a mammalian promoter in the “protected transgene cassette.” Neither the drug selection marker gene, neo, nor the transgene, lacZ, have a mammalian promoter placed upstream from their ORFs. Applicants have previously shown that when this DNA targeting vector is introduced into ES cells, all G418-resistant clones are correctly targeted (see U.S. Ser. No. 09/296,260, filed Jun. 6, 2001, in the name of Regeneron Pharmaceuticals, Inc., and incorporated by reference herein its entirety). Phenotypic evaluation of those targeted ES cell clones showed that, as predicted, none of them stained positive for lacZ. Furthermore, transgenic mice derived from these clones also showed no expression of lacZ. This is because the 4×polyA contains very effective transcription termination signals. When these mice are bred with Cre deletor mice (wherein Cre is expressed at the zygote stage and up to embryonic day 4 in essentially all the cells of the embryo (Williams-Simons and Westphal, 1999, Transgenic Res, 8, 53-4), ubiquitous expression of lacZ (which post-Cre is driven by the ROSA26 promoter, P_{ROSA}) is observed, in agreement with published observations (Soriano, 1999, Nat Genet, 21, 70-1.; Zambrowicz et al., 1997, Proc Natl Acad Sci USA, 94, 3789-94). Therefore, in the absence of an exogenous promoter to drive expression of the transgene, no expression of the transgene is observed. When the transcription termination signal(s) upstream of the transgene are removed, the transgene is expressed ubiquitously, mirroring the expression pattern of the endogenous locus.

Applicants reasoned that placing other exogenous promoters directly upstream of the transgene, which in this example is lacZ, would result in an expression pattern that mirrors that of the exogenous promoter. Several different promoters were tested and the results are summarized in Table 1 and set forth in more detail below.

Since expression of the lacZ in the embodiment described above is silent, Applicants tested whether its expression could be rendered either ubiquitous or restricted to specific cell types by choosing different promoters to drive expression of the transgene. In order to insert only promoter±regulatory elements and to avoid introducing any other variables, Applicants retained lacZ as the transgene. There are three advantages to this: (1) lacz is a very sensitive calorimetric marker and can be visualized easily either in whole mount or sectioned embryos; (2) it has been widely used by other investigators to analyze the expression profiles specified by different promoters and there is a lot of published literature describing this work (Joyner, 1999, The Practical Approach Series, 293); and references within); and (3) it makes possible a direct comparison with the expression profile of lacZ when its expression in driven by the ROSA26 promoter.

### Example 2

The phosphoglycerate kinase (PGK) promoter is a well-established and widely used promoter that confers ubiquitous expression in mice (Adra et al., 1987, Gene, 60, 65-74; McBurney et al., 1994, Dev Dyn, 200, 278-93). It was introduced directly upstream of lacZ in the protected transgene cassette described above and as shown in FIG. 6. The resulting targeting vector is shown in FIG. 7. Note that although the loxP sites have been left in place, there is no intention to use them to remove the neo-4×pA part of the cassette. They have been preserved solely in order to minimize manipulation of and introduction of irrelevant changes in the DNA targeting vector. This DNA targeting vector, containing the protected transgene cassette, was introduced into ES cells and correctly targeted ES cells were used to create transgenic mice. LacZ staining of the targeted ES cells shows that all targeted clones stain positive for lacZ. LacZ staining of chimeric mice whose tissues were in part derived from the targeted ES cells reveals ubiquitous expression of lacZ. Therefore, the PGK promoter was capable of directing ubiquitous expression of a transgene when inserted into the ubiquitously expressed ROSA26 locus and when the transgene was not accessible to transcription from the ROSA26 promoter.

### Example 3

As a next step, Applicants tested several tissue-specific promoters. One such promoter is the insulin promoter that directs expression in the β-cells of the pancreas (Vasavada et al., 1996, J Biol Chem, 271, 1200-8). This promoter was introduced directly upstream of lacZ in the protected transgene cassette described above and as shown in FIG. 6. The resulting DNA targeting vector is shown in FIG. 8. This DNA targeting vector was introduced into ES cells and targeted ES cells were used to create transgenic mice. LacZ staining of the targeted ES cells showed that none of the targeted clones stained positive for lacZ, as would be expected for a promoter that directs expression of a gene only in specific differentiated cells. LacZ staining of chimeric mice whose tissues were in part derived from the targeted ES cells showed that expression of lacZ was restricted to the β-cells of the pancreas. There was no staining in any other tissue. Therefore, a tissue-specific promoter, and particularly one that directs expression essentially only in one cell type, was capable of retaining its specificity when inserted into the ubiquitously expressed ROSA26 locus and when the transgene was not accessible to transcription from the ROSA26 promoter.

In order to show the reproducibility and general applicability of the methods of the invention, similar DNA targeting vectors containing protected transgene cassettes were constructed using other tissue-specific promoters. Table 1 is a summary of the results obtained with the DNA targeting vectors. Note that only the promoters and associated regulatory elements differ. The remaining components and features of the DNA targeting vector remain the same. All the promoters tested displayed the expected expression pattern. These indicate that this methods of the invention are generally applicable.

TABLE 1

| Promoter and | | Predominant |
|---|---|---|
| regulatory | | site of |
| elements | TGN | Expression |
| none | lacZ | None observed |
| Phosphoglycerate | lacZ | Broadly |
| kinase promoter | | expressed |
| (PGK) | | |
| Rat insulin | lacZ | β cells of |
| promoter (RIP) | | the pancreas |
| Human skeletal | lacZ | Skeletal and |
| actin promoter | | cardiac muscle |
| (HSA) | | myocytes |
| Collagen type II | lacZ | Chondrocytes in |
| promoter/enhancer | | Cartilage |
| (a1(II)p-TGN-a1(II)e) | | |
| Smooth Muscle protein | lacZ | Visceral and |
| 22 alpha promoter | | vascular smooth |
| (SM22alpha) | | muscle cells |
| Clara Cell | LacZ | Clara Cells of |
| Promoter 10 (CC10) | | the Respiratory |
| | | Epithelium |
| Keratin 14 | LacZ | Basal |
| (K14) | | Keratinocytes |

Abbreviations:
TGN = transgene
p = promoter
e = enhancer

We claim:

1. A method of expressing a gene of interest in eukaryotic cells, comprising:
a) constructing a DNA targeting vector containing a nucleotide sequence, comprising:
a 5′ homology arm,
a protected transgene cassette, and
a 3′ homology arm,
wherein the protected transgene cassette is comprised of a transcriptional stop signal, an exogenous promoter, and a gene of interest, and wherein the 5′ and 3′ homology arms are derived from a predetermined locus;
b) introducing the DNA targeting vector of (a) into eukaryotic cells such that the targeting vector integrates by homologous recombination into the a predetermined locus; and
c) screening the eukaryotic cells of (b) to identify those cells in which the gene of interest is expressed.

2. A method of genetically modifying a eukaryotic cell by integrating a nucleotide sequence into a predetermined locus, comprising:
a) constructing a DNA targeting vector containing a nucleotide sequence, comprising:
a 5′ homology arm,
a protected transgene cassette, and
a 3′ homology arm,
wherein the protected transgene cassette is comprised of a transcriptional stop signal, an exogenous promoter, and a gene of interest, and wherein the 5′ and 3′ homology arms are derived from the a predetermined locus;
b) introducing the DNA targeting vector of (a) into eukaryotic cells such that the targeting vector integrates by homologous recombination into the a predetermined locus; and
c) screening the eukaryotic cells of (b) to identify those cells that have been genetically modified by integrating a nucleotide sequence into a predetermined locus.

3. A method of integrating a nucleotide sequence into a predetermined locus in eukaryotic cells, comprising:
a) constructing a DNA targeting vector containing a nucleotide sequence, comprising:
a 5′ homology arm,
a protected transgene cassette, and
a 3′ homology arm,
wherein the protected transgene cassette is comprised of a transcriptional stop signal, an exogenous promoter, and a gene of interest, and wherein the 5′ and 3′ homology arms are derived from the predetermined locus;
b) introducing the DNA targeting vector of (a) into eukaryotic cells such that the targeting vector integrates by homologous recombination into the predetermined locus; and
c) screening the eukaryotic cells of (b) to identify those cells in which the nucleotide sequence has integrated by integrating a nucleotide sequence into a predetermined locus.

4. A method of evaluating a gene product's biological activity, comprising:
a) constructing a DNA targeting vector containing a nucleotide sequence, comprising:
a 5′ homology arm,
a protected transgene cassette, and
a 3′ homology arm,
wherein the protected transgene cassette is comprised of a transcriptional stop signal, an exogenous promoter, and a gene of interest, and wherein the 5′ and 3′ homology arms are derived from a predetermined locus;
b) introducing the DNA targeting vector of (a) into eukaryotic cells such that the targeting vector integrates by homologous recombination into the predetermined locus;
c) screening the eukaryotic cells of (b) to identify those cells in which the gene of interest is expressed; and
d) evaluating the gene product's biological activity.

5. A method of evaluating tissue-specific promoter activity, comprising:
a) constructing a DNA targeting vector containing a nucleotide sequence, comprising:
a 5′ homology arm,
a protected transgene cassette, and
a 3′ homology arm,
wherein the protected transgene cassette is comprised of a transcriptional stop signal, an exogenous promoter, and a gene of interest, and wherein the 5′ and 3′ homology arms are derived from a predetermined locus;
b) introducing the DNA targeting vector of (a) into eukaryotic cells such that the targeting vector integrates by homologous recombination into the predetermined locus;
c) screening the eukaryotic cells of (b) to identify those cells in which the gene of interest is expressed; and
d) evaluating the tissue-specific promoter activity.

6. A method of evaluating the activity of the regulatory regions of a gene of interest, comprising:
a) constructing a DNA targeting vector containing a nucleotide sequence, comprising:
a 5′ homology arm,
a protected transgene cassette, and
a 3′ homology arm,
wherein the protected transgene cassette is comprised of a transcriptional stop signal, an exogenous promoter, and a gene of interest, and wherein the 5′ and 3′ homology arms are derived from a predetermined locus;
b) introducing the DNA targeting vector of (a) into eukaryotic cells such that the targeting vector integrates by homologous recombination into the predetermined locus;
c) screening the eukaryotic cells of (b) to identify those cells in which the gene of interest is expressed; and
d) evaluating the activity of the regulatory regions of a gene of interest.

7. The method of claim 1, 2, 3, 4, 5, or 6 wherein the predetermined locus is an ubiquitously expressed locus.

8. The method of claim 7, wherein the ubiquitously expressed locus is the ROSA26 locus.

9. The method of claim 1, 2, 3, 4, 5, or 6 wherein the eukaryotic cell is an embryonic stem cell.

10. The method of claim 9 wherein the embryonic stem cell is a mouse, rat, chicken, rabbit, dog, cat, cow, horse, pig, sheep, or non-primate embryonic stem cell.

11. The method of claim 1, 2, 3, 4, 5, or 6, wherein the protected transgene cassette further comprises neomycin, hygromycin, or puromycin.

12. The method of claim 1, 2, 3, 4, 5, or 6, wherein the protected transgene cassette further comprises additional transcriptional stop signals.

13. The method of claim 11, 2, 3, 4, 5, or 6, wherein the protected transgene cassette further comprises regulatory elements, enhancers, silencers, or insulators.

14. The method of claim 1, 2, 3, 4, 5, or 6, wherein the protected transgene cassette further comprises accessory elements, loxp sites, FRT sites, internal ribosome binding sites (IRES), or operators.

15. The method of claim 1, 2, 3, 4, 5, or 6, wherein the protected transgene cassette further comprises recombinases, repressors or transactivators.

16. The method of claim 15, wherein the recombinases are Cre and FLP, the repressor is the Tetracycline Repressor (TetR), and the transactivator is the Tetracycline Transactivator (tTA).

17. The method of claim 1, 2, 3, 4, 5, or 6, wherein the protected transgene cassette further comprises lacZ, placental alkaline phosphatase, or any member of the fluorescent protein family.

18. A non-human organism containing a genetically modified predetermined locus, wherein the modification is the introduction by homologous recombination into the predetermined locus a nucleotide sequence, comprising:
a 5′ homology arm,
a protected transgene cassette, and
a 3′ homology arm,
wherein the protected transgene cassette is comprised of a transcriptional stop signal, an exogenous promoter, and a gene of interest, and wherein the 5′ and 3′ homology arms are derived from the predetermined locus.

19. The non-human organism of claim 18 wherein the predetermined locus is an ubiquitously expressed locus.

20. The non-human organism of claim 19 wherein the ubiquitously expressed locus is the ROSA26 locus.

21. The non-human organism of claim 18, 19, or 20, which is a mouse.

22. A DNA targeting vector containing a nucleotide sequence, comprising:
a 5′ homology arm,
a protected transgene cassette, and
a 3′ homology arm,
wherein the protected transgene cassette is comprised of a transcriptional stop signal, an exogenous promoter, and a gene of interest, and wherein the 5′ and 3′ homology arms are derived from a predeterrmined locus.

23. The DNA targeting vector of claim 22 wherein the predetermined locus is an ubiquitously expressed locus.

24. The DNA targeting vector of claim 23 wherein the ubiquitously expressed locus is the ROSA26 locus.

25. The DNA targeting vector of claim 22, 23, or 24, wherein the protected transgene cassette further comprises neomycin, hygromycin, or puromycin.

26. The DNA targeting vector of claim 22, 23, or 24, wherein the protected transgene cassette further comprises a transcriptional stop signal sequence.

27. The DNA targeting vector of claim 22, 23, or 24, wherein the protected transgene cassette further comprises regulatory elements, enhancers, silencers, or insulators.

28. The DNA targeting vector of claim 22, 23, or 24, wherein the protected transgene cassette further comprises accessory elements, loxP sites, FRT sites, internal ribosome binding sites (IRES), or operators.

29. The DNA targeting vector of claim 22, 23, or 24, wherein the protected transgene cassette further comprises recombinases, repressors, or transactivators.

30. The DNA targeting vector of claim 29, wherein the recombinases are Cre and FLP recombinases, the repressor is the Tetracycline Repressor (TetR), and the transactivator is the Tetracycline Transactivator (tTA).

31. The DNA targeting vector of claim 22, 23, or 24, wherein the protected transgene cassette further comprises lacZ, placental alkaline phosphatase, or any member of the fluorescent protein family.

32. A cell containing the DNA targeting vector of claim 22, 23, or 24.
